(19) United States
(12) Patent Application Publication
(10) Pub. No.: US 2024/0254510 A1
(43) Pub. Date: Aug. 1, 2024

(54) METHODS FOR GENERATING MECHANICALLY-RESPONSIVE CELLS AND USES THEREOF

(71) Applicants: Washington University, St. Louis, MO (US); Shriners Hospitals for Children, Tampa, FL (US)

(72) Inventors: Farshid Guilak, St. Louis, MO (US); Robert Nims, St. Louis, MO (US); Lara Pferdehirt, St. Louis, MO (US)

(21) Appl. No.: 18/282,761

(22) PCT Filed: Mar. 21, 2022
(86) PCT No.: PCT/US22/21156

§ 371 Date: Sep. 18, 2023

Related U.S. Application Data
(60) Provisional application No. 63/163,528, filed on Mar. 19, 2021.

(51) Int. Cl. C12N 15/86; C07K 14/52; C12N 5/077

(52) U.S. Cl. CPC C12N 15/86; C07K 14/52; C12N 5/0655; C12N 2510/00; C12N 2740/15043; C12N 2830/002

(57) ABSTRACT

Among the various aspects of the present disclosure is the provision of compositions and methods of making genetically modified cells comprising a synthetic circuit that is responsive to a mechanical input to the cell and methods of use thereof. This disclosure uses mechanotransduction to provide gene-based delivery of biologic drugs at prescribed times, phases and frequencies. Once reprogrammed, the cells can be reimplanted in the body for this purpose.

### CROSS REFERENCE TO RELATED APPLICATION

This application claims priority to U.S. Provisional Application No. 63/163,528, filed Mar. 19, 2021, the disclosure of which is incorporated herein by reference.

### FIELD OF THE TECHNOLOGY

This disclosure generally relates to mechanogenetics, cell biology and cellular-mediated therapeutics. Disclosed herein are compositions and methods for cell therapy using engineered cells capable of sensing mechanobiologic stimuli and using mechanical input to drive production of a therapeutic transgene.

### BACKGROUND

Accumulated evidence indicates that mechanical cues, which include physical forces (e.g. tension, compression or shear stress), alterations in extracellular matrix (ECM) mechanics and changes in cell shape, are transmitted to the nucleus to orchestrate transcriptional activities that are crucial for biological responses in cells. The movement of joints, compressive loads on the cartilage and bone during exercise, and shear pressure on the blood vessel during blood circulation are all examples of mechanical forces in human tissues. In addition to the maintenance of normal cell and tissue function, advances in mechanobiology suggest that changes in cell mechanics, extracellular matrix structure, and/or mechanotransduction may contribute to the development of many diseases, including atherosclerosis, fibrosis, asthma, osteoporosis, heart failure, and cancer. There is also a strong mechanical basis for many generalized medical disabilities, such as lower back pain, foot and postural injury, deformity, and irritable bowel syndrome.

Thus, a need exists in the art for synthetic cell-based therapies that sense mechanical stimuli and produce prescribed biologic drugs in response to mechanical stimuli.

### SUMMARY

In an aspect, the present disclosure encompasses a recombinant nucleic acid molecule having at least one transcriptional regulatory nucleic acid sequence of a mechanically-response gene operably linked to a nucleic acid sequence encoding a therapeutic biologic. In some embodiments, the at least one transcriptional regulatory nucleic acid sequence nucleic acid sequence is a promoter. In another aspect, the nucleic acid sequence further comprises a TATA box between the at least one transcriptional regulatory nucleic acid sequence of a mechanically-response gene and the nucleic acid sequence encoding a therapeutic biologic.

In another aspect, the present disclosure provides a nucleic acid construct or vector comprising a nucleic acid molecule at least one transcriptional regulatory nucleic acid sequence of a mechanically-response gene operably linked to a nucleic acid sequence encoding a therapeutic biologic. In some embodiments, the vector is a viral vector. In some embodiments, the viral vector is an adeno-associated viral vector or a lentiviral vector.

In still another aspect, the present disclosure provides a viral particle comprising a viral vector having a nucleic acid molecule with at least one transcriptional regulatory nucleic acid sequence of a mechanically-response gene operably linked to a nucleic acid sequence encoding a therapeutic biologic.

In each of the preceding aspects, the at least one transcriptional regulatory nucleic acid sequence of a mechanically-response gene is a promoter of a mechanically-responsive gene is selected from a NFKB promoter, a PTGS2 promoter, a PMEPA1 promoter, a FGF1 promoter, a SNORA70 promoter, a ADAMTS1 promoter, a IGSF9B promoter, a NR4A2 promoter, a NR4A1 promoter, a INHBA promoter, a CSRNP2 promoter, a PHLDA1 promoter, a GAN promoter, a SLC25A25 promoter, a SLC35E4 promoter, a CAND2 promoter, a SNCAIP promoter, a WNT9A promoter, a EGR1 promoter, a DUSP2 promoter, a SPRY4 promoter and combinations thereof.

In each of the preceding aspects, the therapeutic biologic nucleic acid sequence encodes an anti-catabolic polypeptide, an anti-inflammatory polypeptide, a pro-anabolic polypeptide, a pro-regenerative polypeptide, an anti-microbial polypeptide, an anti-pain polypeptide, a morphogen, a growth factor, an anti-cancer nucleic acid or an anti-cancer polypeptides. In an exemplary embodiment, the promoter is a PTGS2 promoter and the therapeutic biologic is an IL-1 receptor antagonist.

In yet another aspect, the present disclosure provides a genetically modified cell comprising a heterologous nucleic acid sequence incorporated into its genome, wherein the heterologous nucleic acid sequence comprises synthetic circuit having at least one transcriptional regulatory nucleic acid molecule from a mechanically-response gene operably linked to a nucleic acid molecule encoding a therapeutic biologic. In some embodiments, the at least one transcriptional regulatory nucleic acid molecule is a promoter sequence of a mechanically responsive gene. In some embodiments, the promoter of a mechanically-responsive gene is selected from a NFKB promoter, a PTGS2 promoter, a PMEPA1 promoter, a FGF1 promoter, a SNORA70 promoter, a ADAMTS1 promoter, a IGSF9B promoter, a NR4A2 promoter, a NR4A1 promoter, a INHBA promoter, a CSRNP2 promoter, a PHLDA1 promoter, a GAN promoter, a SLC25A25 promoter, a SLC35E4 promoter, a CAND2 promoter, a SNCAIP promoter, a WNT9A promoter, a EGR1 promoter, a DUSP2 promoter, a SPRY4 promoter and combinations thereof. In some embodiments, the nucleic acid sequence further comprises a TATA box between the at least one transcriptional regulatory nucleic acid sequence of a mechanically-response gene and the nucleic acid sequence encoding a therapeutic biologic.

In some embodiments, the therapeutic biologic nucleic acid sequence encodes an anti-catabolic polypeptide, an anti-inflammatory polypeptide, a pro-anabolic polypeptide, a pro-regenerative polypeptide, an anti-microbial polypeptide, an anti-pain polypeptide, a morphogen, a growth factor, an anti-cancer nucleic acid or an anti-cancer polypeptides.

In an exemplary embodiments, the promoter is a PTGS2 promoter and the therapeutic biologic is an IL-1 receptor antagonist.

In each of the preceding embodiments, the genetically modified cell expresses on its surface a mechanically sensitive ion channel. In some embodiments, the mechanically sensitive ion channel is from the transient receptor potential (TRP) family. In some embodiments, the mechanically sensitive ion channel is selected from TRPA1, TRP vanilloid 1 (TRPV1), and TRPV4. In a specific embodiment, the mechanically sensitive ion channel is TRPV4. In an exemplary embodiment, when the mechanically sensitive ion channel is TRPV4, the promoter of a mechanically-responsive gene is selected from a NF-KB promoter, a PTGS2 promoter, a PMEPA1 promoter, a FGF1 promoter, a IL11 promoter, a LAMC2 promoter, a LAMA3 promoter, a HBEGF promoter, a JUNB promoter, a ATF3 promoter, a INHBA promoter, a CCN1 promoter, a NGF promoter, a TGFB1 promoter, a ERF promoter, a FOS promoter, a KLF4 promoter, a TEAD4 promoter, a TNFRSF11B promoter, and combinations thereof and the therapeutic biologic nucleic acid molecule encodes for one or more of IL-1Ra, sTNFR1/2, IL-10 IL-4, a growth factor from the TGFβ superfamily, IGF, CTGF, FGF, PDGF, TNF, IL-7, IL-15, IL-12, IL-2, IFN, NOS, PTGIS, Decorin, TGFβ-receptor, MMP, ALDH2, NR3C1 and any combination thereof.

In another exemplary embodiment, the mechanically sensitive ion channel is PIEZO1 and/or PIEZO2 and the promoter of a mechanically-responsive gene is selected from a ADAMTS1 promoter, a NR4A2 promoter, a NR4A1 promoter, a WNT9A promoter, a SPRY4 promoter, and combinations thereof and the therapeutic biologic nucleic acid molecule encodes for one or more of IL-1Ra, sTNFR1/2, IL-10 IL-4, a growth factor from the TGFβ superfamily, IGF, CTGF, FGF, PDGF, a kappa opioid ligand pro-peptide (e.g., prodynorphin), a mu/delta opioid ligand pro-peptide (e.g., proenkephalin), a delta/mu opioid ligand pro-peptide (proopiomelanocortin), an endocannabinoid ligand synthesis drivers TNF, IL-7, IL-15, IL-12, IL-2, IFN, NOS, PTGIS, Decorin, TGFβ-receptor, MMP, ALDH2, NR3C1 and any combination thereof.

In each of the above embodiments, the cell can be autologous or allogeneic and a somatic cell or a stem cell. In each of the above embodiments, the period, frequency, or phase of the therapeutic biologic expression is modulated through the at least one transcriptional regulatory nucleic acid molecule of a mechanically responsive gene or through use of combinations of the transcriptional regulatory nucleic acid molecules of a mechanically responsive gene.

In some embodiments, the genetically modified cells is selected from an embryonic stem cells (ES), a somatic adult stem cell, a tissue-specific stem cell, an induced pluripotent stem cells (iPSCs), a progenitor cell, a fibroblast, a cardiomyocyte, a hepatocyte, an endothelial cell, a chondrocyte, a smooth muscle cell, a striated muscle cells, a bone cell, a synovial cell, a tendon cell, a ligament cell, a meniscus cell, an adipose cell, a splenocyte, an epithelial cell, a melanocyte, a neuron, an astrocyte, a microglial cell, a vascular cell, a B-cells, a dendritic cell, a natural killer cell, or a T-cell.

In another aspect, the present disclosure provides an implantable tissue comprising a genetically modified cell of the disclosure and/or a pharmaceutical composition comprising the genetically modified cell of the disclosure.

In still another aspect, the present disclosure provides a method of treating a condition, disease or disorder in a subject in need thereof, the method comprising administering an effective amount of a composition comprising a genetically modified cell of the disclosure. In some embodiments, the condition, disease or disorder is a chronic inflammatory disease, an acute inflammatory disease, a degenerative disease of any tissue or organ, chronic or acute pain, cancer, cardiovascular disease, osteoarthritis, cardiac hypertrophy, atherosclerosis, asthma, irritable bowel syndrome, muscle atrophy, angina, atrial fibrillation, hypertension, intimal hyperplasia, valve disease, scleroderma, achalasia, volvulus, diabetic nephropathy, glomerulosclerosis, cerebral edema, hydrocephalus, migraine, stroke, glaucoma, ankylosing spondylitis, carpal tunnel syndrome, chronic back pain, dupytre's contracture, osteoporosis, rheumatoid arthritis, collagenopathies, Musculo dystrophies, osteochondroplasias, polycystic kidney disease, ARDS, emphysema, pulmonary fibrosis, ventilator injury, pre-eclampsia, sexual dysfunction, and urinary incontinence.

Other aspects and iterations of the invention are described more thoroughly below.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts an exemplary mechanogenetic transduction and therapeutic drug delivery approach. TRPV4 is an osmotically sensitive cation channel in the cell membrane of chondrocytes, which can be activated by mechanical loading secondary to mechano-osmotic coupling through the extracellular matrix or pharmacologically with the agonist GSK101. TRPV4 can also be inhibited with the antagonist GSK205. Upon TRPV4 activation, chondrocytes respond with intracellular calcium signaling that initiates NF-κB signaling and up-regulation of the PTGS2 gene. By lentivirally transducing synthetic mechanogenetic circuits that respond to either NF-κB activation or PTGS2 up-regulation into chondrocytes within an engineered cartilage tissue, mechanically activated TRPV4 signaling was used to drive transgene production of either a luciferase reporter or the therapeutic anti-inflammatory biologic IL-1Ra. GOI; gene of interest.

FIG. 2A, FIG. 2B, FIG. 2C, FIG. 2D, FIG. 2E, FIG. 2F, and FIG. 2G show mechanical responsiveness of chondrocytes is mediated by hypo-osmotic stimulation of TRPV4. FIG. 2A shows the setup of real-time cellular imaging of mechanical loading. Loading chondrocytes within engineered cartilage increases intracellular calcium compared to free swelling. Arrows indicate immediately responsive cells. Scale bar, 50 μm. The number of cells exhibiting intracellular calcium signaling increased by 108% after loading, and GSK205 suppressed cellular calcium signaling (n=4 to 5 constructs per treatment). TE, tissue engineered. FIG. 2B shows isolated chondrocytes are sensitive to osmotic perturbations and exhibit intracellular calcium increases in response to hypo-osmotic stimulation (n=15 to 20 cells per group, calcium response is normalized to calcium levels at 354 mOsm). FIG. 2C shows chondrocyte responsiveness to hypo-osmotic stimulation is inhibited with GSK205 (n=6 per treatment). RFU, relative fluorescence units. FIG. 2D show chondrocytes are not sensitive to direct membrane stretch applied under iso-osmotic, iso-volumetric conditions with micropipette aspiration (n=38; scale bar, 10 μm). FIG. 2E show direct cellular compression under a 400-nN load with an AFM induces intracellular calcium signaling. Scale bar, 10 μm. FIG. 2F shows GSK205 does not modulate calcium response of chondrocytes to AFM loading. FIG. 2G shows TRPV4 inhibition alters neither the intensity of calcium responsiveness nor the population response to AFM compression (n=23 to 30 cells per group). Data are presented as means±SEM.

FIG. 3A, FIG. 3B, FIG. 3C, FIG. 3D, FIG. 3E and FIG. 3F show the transcriptomic profile induced by TRPV4 activation. FIG. 3A shows engineered cartilage tissue constructs were made from isolated primary porcine chondrocytes cast into agarose hydrogels. Tissue constructs were cultured in nutrient-rich medium before deformational mechanical loading or GSK101 pharmacologic stimulation (red, 3 hours per round) following the indicated time course and cartilage construct harvest (arrows). FIG. 3B shows forty-one genes were differentially up-regulated in response to TRPV4 activation, and levels returned back to baseline after 12 to 20 hours after loading (n=3 per treatment/time point). FIG. 3C shows CAMP- and calcium-responsive transcription factors were immediately and highly regulated by both mechanical loading and GSK101 stimulation (red arrows indicate removal from loading). FIG. 3D shows pathway analysis based on transcription activity suggests that both inflammatory and anabolic pathways are strongly regulated by TRPV4 activation. FIG. 3E shows analysis of gene target response after all bouts of mechanical loading and all bouts of GSK101 stimulation produces a list of distinctly TRPV4-sensitive genes. FIG. 3F shows TRPV4-responsive targets from the microarray analysis were confirmed by qPCR (n=2 to 3), and a one-tailed t test was used to test whether loaded or GSK101 groups were significantly up-regulated with treatment. *P<0.05.

FIG. 4A, FIG. 4B, FIG. 4C, FIG. 4D, FIG. 4E, FIG. 4F, FIG. 4G, FIG. 4H, FIG. 4I and FIG. 4J show exemplary mechanogenetic constructs respond to TRPV4 activation. FIG. 4A shows mechanical loading, osmotic loading, or GSK101 stimulation was applied to mechanogenetic tissues; GSK205 inhibits TRPV4 activation. FIG. 4B shows NFKBr-IL1Ra tissues respond to mechanical loading through increased IL-1Ra (P<0.001). IL-1Ra is reduced with GSK205 supplementation (P<0.001, n=6 per treatment). FIG. 4C shows exposure of NFKBr-IL1Ra to hypo-osmotic medium produces more IL-1Ra than iso-osmotic medium exposure (P=0.019, n=7 per group). FIG. 4D shows NFKBr-IL1Ra tissues exposed to GSK101 stimulation produce more IL-1Ra than vehicle controls (P<0.001, n=20 per group). FIG. 4E shows mechanical loading of NFKBr-Luc tissues quickly activates and inactivates circuits, while PTGS2r-Luc tissues take longer to reach the peak and return to baseline (gray line denotes P<0.05 between free swelling and load, n=3 to 6 per group). RLU, relative luminescence units. FIG. 4F shows NFKBr-IL1Ra tissue response to loading after 24 and 72 hours, indicating differential expression in first 24 hours. n.s., not significant. FIG. 4G shows PTGS2r-IL1Ra tissues respond to loading through 72 hours. FIG. 4H shows NFKBr-Luc tissues respond dose dependently to TRPV4 activation via GSK101 through 9 nM GSK101 (P<0.05, n=2 to 4 per group). AUC, area under the curve. Different letters denote statistical differences. FIG. 4I shows PTGS2r-Luc tissues are sensitive to GSK101 up to 6 nM (P<0.05, n=2 to 4 per group). FIG. 4J shows NFKBr-IL1Ra tissue response is dose dependent to compressive mechanical loading strain from 0 to 15% (P<0.001, n=5 to 12). Data are presented as means±SEM. *P<0.05.

FIG. 5A, FIG. 5B, FIG. 5C, FIG. 5D, FIG. 5E, FIG. 5F, FIG. 5G, FIG. 5H, FIG. 5I, FIG. 5J, FIG. 5K, FIG. 5L, and FIG. 5M show activation of TRPV4 via osmotic loading of mechanogenetic constructs protects against IL-1a. FIG. 5A shows inflammatory response of NFKBr-IL1Ra constructs under IL-1a supplementation. FIG. 5B shows NFKBr-IL1Ra tissues produce IL-1Ra in response to IL-1a (n=3, P<0.001). FIG. 5C shows inflammatory response of PTGS2r-IL1Ra tissues under IL-1a supplementation. FIG. 5D shows PTGS2r-IL1Ra tissues do not respond to IL-1a (n=5). FIG. 5E shows PTGS2r-Luc tissues that are not altered by IL-1a are modulated by chronic GSK101 (n=2 per condition, arrow indicates stimulation). FIG. 5F shows osmo-inflammatory response of NFKBr-Luc tissues using osmotic loading (3 hours/day) and IL-1a (0 or 0.1 ng/ml) applied to NFKBr-Luc tissues. FIG. 5G shows NFKBr-Luc tissues do not produce IL-1Ra (n=7). n.d., not detectable. FIG. 5H shows NFKBr-Luc tissues lost S-GAG in the presence of IL-1a (n=5 per group). FIG. 5I shows histologically reduced safranin-O staining present with IL-1a supplementation. FIG. 5J shows osmo-inflammatory response of NFKBr-IL1Ra tissues using osmotic loading (3 hours/day) and IL-1a (0 or 0.1 ng/ml). FIG. 5K shows IL-1Ra was increased with inflammation and osmotic loading (n=7, P<0.001). FIG. 5L shows S-GAG content in NFKBr-IL1Ra tissues with IL-1a supplementation (0 or 0.1 ng/ml) and/or osmotic loading (different letters denote significant differences; P<0.05, n=5). FIG. 5M shows NFKBr-IL1Ra tissues displayed similar safranin-O staining without IL-1a supplementation, while IL-1a supplementation reduced safranin-O staining in iso-osmotic tissues but not hypo-osmotic tissues. Data are presented as means±SEM.

FIG. 6 shows RNA-Seq Reveals temporally responsive genes vary between different treatments.

FIG. 7 shows isolating a mechanically-regulated and TRPV4-specific gene target for mechanogenetic circuits.

FIG. 8 shows FGF1 mechanogenetic circuits require endogenous FGF1 promotor for TRPV4 stimulation.

FIG. 9 shows transcriptomic regulation of engineered cartilage tissues to Piezo1 activation.

FIG. 10 shows gene targets similarly regulated by mechanical loading and Yoda1 stimulation points to Piezo1 regulation.

### DETAILED DESCRIPTION

The present disclosure provides compositions, systems and methods for cellular therapy comprising a genetically modified mechanically-responsive cell. In particular, target cells are modified with a synthetic circuit which drives expression of a biologic therapeutic (e.g., a therapeutic polypeptide or RNA molecule). The synthetic circuit allows for activation by a prescribed input to produce the delivery of the biologic therapeutic in a controlled manner to enhance the therapeutic effect of various therapies, such as stem cell therapies, for tissue regeneration and treatment of a variety of acute and chronic diseases and cancer.

The present disclosure is based, at least in part, on the deconstruction of the signaling networks induced by activation of mechanically sensitive ion channels. Synthetic circuits were then created and incorporated into the genome of cells which express the mechanically sensitive ion channel. These cells are then engineered into living tissues that are able to respond to mechanical input by activating the ion channel-mediated intercellular signaling to target the synthetic circuit resulting in transcription of the biologic therapeutic. For example, the present disclosure shows either osmotic or mechanical loading of chondrocytes transduced with transient receptor potential vanilloid 4 (TRPV4)-responsive synthetic circuits protected tissue incorporating the transduced cells from inflammatory degradation by interleukin-1α through the expression of the anti-inflammatory biologic drug, interleukin-1 receptor antagonist.

Other aspects and iterations of the invention are described more thoroughly below.

### I. Compositions

Provided herein are compositions for use in cellular therapy. The compositions including a synthetic circuit which comprises, consist essentially of, or consist of an engineered nucleic acid sequence having at least a transcriptional regulatory region (e.g. a promoter) of a mechanically-responsive gene operably linked to a nucleic acid sequence encoding a biologic therapeutic (e.g., a therapeutic polypeptide or RNA molecule). In some embodiments, the disclosure provides the synthetic circuit incorporated into a plasmid or vector, such as an expression vector and/or viral vector.

In one aspect, the disclosure provides a genetically modified cell having a synthetic circuit of the disclosure. In some aspects, the regulatory region and nucleic acid sequence encoding the biologic therapeutic are recombinant to the cell. For example, the synthetic circuit can be used to create a genetically modified cell using a plasmid, viral transduction, or gene editing.

The genetically modified cell may be any autologous or allogeneic somatic or stem cell (e.g., adult, embryonic, or induced pluripotent stem cell). The genetically modified target cell may be endogenous or exogenous, and may be genetically modified in situ or ex vivo. Alternatively, the genetically modified target cell maybe be genetically modified ex situ, and then reimplanted in the body as a cell therapy or engineered into an implantable tissue. Thus, the present disclosure provides compositions comprising the genetically modified cells.

A composition of the disclosure may optionally comprise one or more additional drug or therapeutically active agent in addition to the genetically modified cells. A composition of the disclosure may further comprise a pharmaceutically acceptable excipient, carrier, or diluent. Further, a composition of the disclosure may contain preserving agents, solubilizing agents, stabilizing agents, wetting agents, emulsifiers, salts (substances of the present invention may themselves be provided in the form of a pharmaceutically acceptable salt), buffers, coating agents, or antioxidants.

### a) Synthetic Circuit

As used herein, a “synthetic circuit” refers to a nucleic acid sequence comprising at least one transcriptional regulatory region from one or more mechanically-responsive genes such that the regulatory region is operably linked to a nucleic acid sequence encoding a therapeutic biologic. As used herein, a “mechanically-responsive gene” refers to a reference endogenous gene that is transcriptionally regulated (completely or partially) by an initial mechanical input to a cell comprising the endogenous gene, and results in signal transduction (i.e. mechanotransduction) in the cell (e.g. activation of a cellular sensor of the mechanical input leading to the regulation of activity of one or more transcription factor(s) specific to the gene) and increased expression of the coding region of the gene.

The mechanical input can be, in non-limiting examples, a hydrostatic pressure, a shear stress, a compressive force, a tensional force, a cell traction force, and/or a cell prestress, and the mechanical input can be generated by any natural or artificial source. A hydrostatic pressure refers to mechanical force applied by fluids or gases (e.g. blood or air) that perfuse or infuse living organs (e.g. blood vessels or lung). A shear stress refers to a frictional force of fluid flow on the surface of cells. For example, the shear stress generated by the heart pumping blood through the systemic circulation has a key role in the determination of the cell fate of cardiomyocytes, endothelial cells and hematopoietic cells. A compressive force refers to a pushing force that shortens the material in the direction of the applied force. A tensional force refers to a pulling force that lengthens materials in the direction of the applied force. A cell traction force refers to a force that is exerted on the adhesion to the ECM and other cells as a result of the shortening of the contractile cytoskeletal actomyosin filaments, which transmit tensional forces across cell surface adhesion receptors (e.g. integrins, cadherins). A cell prestress is a stabilizing isometric tension in the cell that is generated by the establishment of a mechanical force balance within the cytoskeleton through a tensegrity mechanism. For example, pulling forces generated within contractile microfilaments are resisted by external tethers of the cell (e.g. to the ECM or neighboring cells) and by internal load-bearing structures that resist compression (e.g. microtubules, filipodia). Prestress controls signal transduction and regulates cell fate.

In an exemplary embodiment, a cellular sensor of a mechanical input is a mechanically sensitive ion channel. Mechanically sensitive ion channels respond to a mechanical input by altering their conformation between an open state and a closed state thereby initiating mechanotransduction. In some embodiments, the mechanically sensitive ion channel is a mechanically sensitive ion channel from the transient receptor potential (TRP) family. Thus, in some embodiments, the mechanically sensitive ion channel is selected from TRPA1, TRP vanilloid 1 (TRPV1), and TRPV4. As another non-limiting example, the mechanically sensitive ion channel is a mechanically sensitive ion channel from the PIEZO family. Thus, in some embodiments, the mechanically sensitive ion channel is selected from PIEZO1 and PIEZO2. Thus, the present disclosure provides for compositions and methods for transducing a cell expressing on its surface a functional cellular sensor of a mechanical input (e.g. a mechanically sensitive ion channel) with a synthetic circuit where the at least one transcriptional regulatory region from one or more mechanically-responsive genes provided in the synthetic circuit is responsive to the specific mechanotransduction mediated by said cellular sensor

Thus, the present disclosure provides a synthetic circuit comprising one transcriptional regulatory regions from one or more mechanically-responsive genes (e.g. promoters). A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

In one embodiment, the transcriptional regulatory region is one or more nucleic acid sequences corresponding to a promoter region of a mechanically responsive gene. In one aspect, the nucleic acid sequence corresponds to a promoter region of a PTGS2 gene (Prostaglandin-Endoperoxide Synthase 2)(e.g., a promoter as designated by GeneCard ID: GC01M186640; chr1: 186,671,791-186,680,922 (GRCh38/hg38); GeneHancer Identifier GH01J186678 (i.e. chr1:186678001-186683001 (GRCh38/hg38); GeneHancer Identifier GH01J186654 (i.e. chr1:186652601-186661400 (GRCh38/hg38); GeneHancer Identifer GH01J186643 (i.e. chr1:186643401-186644800 (GRCh38/hg38); GeneHancer Identifer GH01J186617 (i.e. chr1:186617934-186619111 (GRCh38/hg38); and/or GeneHancer Identifier GH01J186738 (i.e. chr1:186738240-186739600 (GRCh38/hg38)); a promoter region of a PMEPA1 gene (Prostate Transmembrane Protein, Androgen Induced 1)(e.g., a promoter as provided by GeneCard ID: GC20M057648; chr20:57,648,392-57,711,536 (GRCh38/hg38); GeneHancer Identifier GH20J057706 (i.e. chr20:57705800-57711626 (GRCh38/hg38)); GeneHancer Identifier GH20J057712 (i.e. chr20:57712400-57713001 (GRCh38/hg38)); Gene Hancer Identifier GH20J057747 (i.e. chr20:57747707-57750152 (GRCh38/hg38)); GeneHancer Identifier GH20J057691 (i.e. chr20:57691639-57700490 (GRCh38/hg38)); and/or GeneHancer Identifier GH20J057807 (i.e. chr20:57807801-57812399 (GRCh38/hg38)); a promoter region of a FGF1 gene (Fibroblast Growth Factor 1)(e.g., a promoter as designated by GeneCard ID: GC05M142555; chr5:142,592, 178-142,698,070 (GRCh38/hg38); GeneHancer Identifier GH05J142684 (i.e. chr5:142684021-142687034 (GRCh38/hg38)); GeneHancer Identifier GH05J142695 (i.e. chr5:142695988-142699365 (GRCh38/hg38)); GeneHancer Identifier GH05J142620 (i.e. chr5:142620772-142622904 (GRCh38/hg38)); GeneHancer Identifier GH05J142680 (i.e. chr5:142680201-142681952 (GRCh38/hg38)); and/or GeneHancer Identifier GH05J142673 (i.e. chr5:142673449-142676891 (GRCh38/hg38)); a promoter region of a IL11 gene (Interleukin 11)(e.g., a promoter as designated by GeneCard ID: GC19M055364; chr19:55,364,382-55,370,463 (GRCh38/hg38); GeneHancer Identifier GH19J055382 (i.e. chr19:55382400-55390955 (GRCh38/hg38)); GeneHancer Identifier GH19J055368 (i.e. chr19:55368544-55371436 (GRCh38/hg38)); GeneHancer Identifier GH19J055375 (i.e. chr19:55375841-55378201 (GRCh38/hg38)); GeneHancer Identifier GH19J055337 (i.e. chr19:55337821-55342001 (GRCh38/hg38)); and/or GeneHancer Identifier GH19J055459 (i.e. chr19:55459434-55463001 (GRCh38/hg38)); a promoter region of a LAMC2 gene (Laminin Subunit Gamma 2)(e.g., a promoter as designated by GeneCard ID: GC01P183186; chr1:183, 186,238-183,245, 127 (GRCh38/hg38); GeneHancer Identifier GH01J183184 (i.e. chr1:183184416-183189818 (GRCh38/hg38)); GeneHancer Identifier GH01J183158 (i.e. chr1:183158801-183166780 (GRCh38/hg38)); GeneHancer Identifier GH01J183190 (i.e. chr1:183190860-183201000 (GRCh38/hg38)); GeneHancer Identifier GH01J183233 (i.e. chr1:183233935-183238999 (GRCh38/hg38)); and/or GeneHancer Identifier GH01J183217 (i.e. chr1:183217532-183220599 (GRCh38/hg38)); a promoter region of a LAMA3 gene (Laminin Subunit Alpha 3)(e.g., a promoter as designated by GeneCard ID: GC18P023689; chr18:23,689,443-23,956,222 (GRCh38/hg38); GeneHancer Identifier GH18J023688 (i.e. chr18:23688800-23691601 (GRCh38/hg38)); GeneHancer Identifier GH18J023870 (i.e. chr18:23870338-23877646 (GRCh38/hg38)); GeneHancer Identifier GH18J023882 (i.e. chr18:23882510-23887399 (GRCh38/hg38)); GeneHancer Identifier GH18J023662 (i.e. chr18:23662000-23664200 (GRCh38/hg38)); and/or GeneHancer Identifier GH18J023715 (i.e. chr18:23714602-23722786 (GRCh38/hg38)); a promoter region of a HBEGF gene (Heparin Binding EGF Like Growth Factor)(e.g., a promoter as designated by GeneCard ID: GC05M140332; chr5:140,332,843-140,346,603 (GRCh38/hg38); GeneHancer Identifier GH05J140341 (i.e. chr5:140341121-140349992 (GRCh38/hg38)); GeneHancer Identifier GH05J140257 (i.e. chr5:140257390-140262885 (GRCh38/hg38)); GeneHancer Identifier GH05J140321 (i.e. chr5:140321936-140325724 (GRCh38/hg38)); GeneHancer Identifier GH05J140301 (i.e. chr5:140301200-140304001 (GRCh38/hg38)); and/or GeneHancer Identifier GH05J140269 (i.e. chr5:140269729-140276800 (GRCh38/hg38)); a promoter region of a JUNB gene (JunB Proto-Oncogene, AP-1 Transcription Factor Subunit)(e.g., a promoter as designated by GeneCard ID: GC19P012791; chr19: 12,791,486-12,793,315 (GRCh38/hg38); GeneHancer Identifier GH19J012774 (i.e. chr19:12774312-12797277 (GRCh38/hg38)); GeneHancer Identifier GH19J012609 (i.e. chr19:12609200-12614750 (GRCh38/hg38)); GeneHancer Identifier GH19J013148 (i.e. chr19:13148000-13158783 (GRCh38/hg38)); GeneHancer Identifier GH19J013160 (i.e. chr19:13160386-13174204 (GRCh38/hg38)); and/or GeneHancer Identifier GH19J012757 (i.e. chr19:12757331-12758395 (GRCh38/hg38)); a promoter region of a ATF3 gene (Activating Transcription Factor 3)(e.g., a promoter as designated by GeneCard ID: GC01P212565; chr1:212,565,334-212,620,777 (GRCh38/hg38); GeneHancer Identifier GH01J212605 (i.e. chr1:212605186-212611201 (GRCh38/hg38)); GeneHancer Identifier GH01J212565 (i.e. chr1:212565996-212568199 (GRCh38/hg38)); GeneHancer Identifier GH01J212615 (i.e. chr1:212615214-212615273 (GRCh38/hg38)); GeneHancer Identifier GH01J212590 (i.e. chr1:212590338-212590397 (GRCh38/hg38)); and/or GeneHancer Identifier GH01J212481 (i.e. chr1:212481825-212490008 (GRCh38/hg38)); a promoter region of a INHBA gene (Inhibin Subunit Beta A)(e.g., a promoter as designated by GeneCard ID: GC07M041668; chr7:41,667, 168-41,710,532 (GRCh38/hg38); GeneHancer Identifier: GH07J041694 (i.e. chr7:41694761-41707219 (GRCh38/hg38)); GeneHancer Identifier: GH07J041708 (i.e. chr7:41708743-41710423 (GRCh38/hg38); GeneHancer Identifier: GH07J41711 (i.e. chr7:41711601-41712033 (GRCh38/hg38); GeneHancer Identifier: GH07J40823 (i.e. chr7:40823724-40828429 (GRCh38/hg38); and/or GeneHancer Identifier: chr7:40807275-40811399 (GRCh38/hg38)); a promoter region of a CCN1 gene (Cellular Communication Network Factor 1)(e.g., a promoter as designated by GeneCard ID: GC01P085581; chr1:85,580,761-85,584,589 (GRCh38/hg38); GeneHancer Identifier: GH01J085575 (i.e. chr7:41694761-41707219 (GRCh38/hg38)); GeneHancer Identifier: GH01J085604 (i.e. chr1:85604570-85614535 (GRCh38/hg38); GeneHancer Identifier: GH01J085520 (i.e. chr1:85520315-85528000 (GRCh38/hg38); GeneHancer Identifier: GH01J085459 (i.e. chr1:85459392-85466601 (GRCh38/hg38); and/or GeneHancer Identifier: GH01J085706 chr1:85706200-85709617 (GRCh38/hg38)); a promoter region of a NGF gene (Nerve Growth Factor)(e.g., a promoter as designated by GeneCard ID: GC01M115285; chr1:115,285,904-115,338,770 (GRCh38/hg38); GeneHancer Identifier: GH01J115337 (i.e. chr1:115336800-115338849 (GRCh38/hg38)); GeneHancer Identifier: GH01J114754 (i.e. chr1:114754394-114759176 (GRCh38/hg38); GeneHancer Identifier: GH01J115425 (i.e. chr1:115425402-115432999 (GRCh38/hg38); GeneHancer Identifier: GH01J114715 (i.e. chr1:114715200-114717632 (GRCh38/hg38); and/or GeneHancer Identifier: GH01J115188 chr1:115188601-115191999 (GRCh38/hg38)); a promoter region of a TGFβ1 gene (Transforming Growth Factor Beta 1)(e.g., a promoter as designated by GeneCard ID: GC19M041301; chr19:41,301,587-41,353,922 (GRCh38/hg38); GeneHancer Identifier: GH19J041348 (i.e. chr19:41348150-41355076 (GRCh38/hg38)); GeneHancer Identifier: GH19J041294 (i.e. chr19:41294892-41311801 (GRCh38/hg38); GeneHancer Identifier: GH19J041321 (i.e. chr19:41321104-41329499 (GRCh38/hg38); GeneHancer Identifier: GH19J041261 (i.e. chr19:41261200-41268475 (GRCh38/hg38); and/or GeneHancer Identifier: GH19J041424 (i.e. chr19:41424528-41429495 (GRCh38/hg38)); a promoter region of a ERF gene (ETS2 Repressor Factor)(e.g., a promoter as designated by GeneCard ID: GC19M042247; chr19:42,247,569-42,255, 128 (GRCh38/hg38); GeneHancer Identifier: GH19J042249 (i.e. chr19:42249279-42257232 (GRCh38/hg38)); GeneHancer Identifier: GH19J042248 (i.e. chr19:42248194-42248253 (GRCh38/hg38); GeneHancer Identifier: GH19J041294 (i.e. chr19:41294892-41311801 (GRCh38/hg38); GeneHancer Identifier: GH19J041957 (i.e. chr19:41957194-41960201 (GRCh38/hg38); and/or GeneHancer Identifier: GH19J042301 (i.e. chr19:42301000-42303848 (GRCh38/hg38)); a promoter region of a FOS gene (Fos Proto-Oncogene, AP-1 Transcription Factor Subunit)(e.g., a promoter as designated by GeneCard ID: GC14P075278; chr14:75,278,826-75,282,230 (GRCh38/hg38); GeneHancer Identifier: GH14J075273 (i.e. chr14:75273233-75285010 (GRCh38/hg38)); GeneHancer Identifier: GH14J075293 (i.e. chr14:75293004-75299870 (GRCh38/hg38); GeneHancer Identifier: GH14J074608 (i.e. chr14:74608801-74622483 (GRCh38/hg38); GeneHancer Identifier: GH14J075249 (i.e. chr14:75249038-75261045 (GRCh38/hg38); and/or GeneHancer Identifier: GH14J075333 (i.e. chr14:75333588-75336192 (GRCh38/hg38)); a promoter region of a KLF4 gene (Kruppel Like Factor 4)(e.g., a promoter as designated by GeneCard ID: GC09M107484; chr9:107,484,852-107,490,482 (GRCh38/hg38); GeneHancer Identifier: GH09J107484 (i.e. chr9:107484240-107492635 (GRCh38/hg38)); GeneHancer Identifier: GH09J107513 (i.e. chr9:107512801-107514599 (GRCh38/hg38)); GeneHancer Identifier: GH09J107754 (i.e. chr9:107754001-107756621 (GRCh38/hg38)); GeneHancer Identifier: GH09J107636 (i.e. chr9:107636660-107640146 (GRCh38/hg38)); and/or GeneHancer Identifier: GH09J107631 (i.e. chr9:107629801-107634400 (GRCh38/hg38)); a promoter region of a TEAD4 gene (TEA Domain Transcription Factor 4)(e.g., a promoter as designated by GeneCard ID: GC12P002959; chr12:2,959,330-3,040,676 (GRCh38/hg38); GeneHancer Identifier: GH12J002957 (i.e. chr12:2957959-2962091 (GRCh38/hg38)); GeneHancer Identifier: GH12J002997 (i.e. chr12:2997860-3001136 (GRCh38/hg38)); GeneHancer Identifier: GH12J003001 (i.e. chr12:3001961-3005372 (GRCh38/hg38)); GeneHancer Identifier: GH12J002980 (i.e. chr12:2980889-2983744 (GRCh38/hg38)); and/or GeneHancer Identifier: GH12J002974 (i.e. chr12:2974209-2975261 (GRCh38/hg38)); a promoter region of a TNFRSF11B gene (TNF Receptor Superfamily Member 11b)(e.g., a promoter as designated by GeneCard ID: GC08M118923; chr12:2,959,330-3,040,676 (GRCh38/hg38); GeneHancer Identifier: GH08J118948 (i.e. chr8:118947600-118952979 (GRCh38/hg38)); GeneHancer Identifier: GH08J118953 (i.e. chr8:118953000-118953200 (GRCh38/hg38)); GeneHancer Identifier: GH08J118876 (i.e. chr8:118876411-118882799 (GRCh38/hg38)); GeneHancer Identifier: GH08J118743 (i.e. chr8:118743262-118746900 (GRCh38/hg38)); and/or GeneHancer Identifier: GH08J118908 (i.e. chr8:118908903-118915140 (GRCh38/hg38)); a promoter region of a SNORA70 (Small Nucleolar RNA, H/ACA Box 70)(e.g., a promoter as designated by GeneCard ID: GC0XP154400; chrX:154,400,281-154,400,415 (GRCh38/hg38); GeneHancer Identifer: GH0XJ154396 (i.e. chrX:154396385-154402574 (GRCh38/hg38)); GeneHancer Identifer: GH0XJ153900 (i.e. chrX: 153900410-153905401 (GRCh38/hg38)); GeneHancer Identifier: GH0X154303 (i.e. chrX:154296801-154315000 (GRCh38/hg38)); GeneHancer Identifier: GH0XJ154723 (i.e. chrX:154723436-154726463 (GRCh38/hg38)); and/or GeneHancer Identifier: GH0XJ153965 (i.e. chrX:153965500-153975531 (GRCh38/hg38)); a promoter region of a ADAMTS1 gene (ADAM Metallopeptidase With Thrombospondin Type 1 Motif 1)(e.g., a promoter as designated by GeneCard ID: GC21M026835; chr21:26, 835, 755-26, 845,409 (GRCh38/hg38); GeneHancer Identifier: GH21J026839 (i.e. chr21:26839809-26847812 (GRCh38/hg38)); GeneHancer Identifier: GH21J026961 (i.e. chr21:26961861-26968600 (GRCh38/hg38)); GeneHancer Identifier: GH21J026813 (i.e. chr21:26813585-26816059 (GRCh38/hg38); GeneHancer Identifier: GH21J026788 (i.e. chr21:26788401-26790000 (GRCh38/hg38)); and/or GeneHancer Identifier: GH21J026800 (i.e. chr21:26798944-26802919 (GRCh38/hg38)); a promoter region of a IGSF9B gene (Immunoglobulin Superfamily Member 9B)(e.g., a promoter as designated by GeneCard ID: GC11M133969; chr11:133,896,438-133,956,968 (GRCh38/hg38); GeneHancer Identifier: GH11J13395; GeneHancer Identifier: GH11J133946; GeneHancer Identifier: GH11J134032; and/or GeneHancer Identifier: GH11J133950)); a promoter region of a NR4A2 gene (Nuclear Receptor Subfamily 4 Group A Member 2)(e.g., a promoter as designated by GeneCard ID: GC02M156324; chr2:156,324,432-156,342,348 (GRCh38/hg38); GeneHancer Identifier: GH02J156331 (i.e. chr2:156330400-156336298 (GRCh38/hg38)); GeneHancer Identifier: GH02J156338 (i.e. chr2:156338128-156344406 (GRCh38/hg38)); GeneHancer Identifier: GH02J156434 (i.e. chr2:156434658-156439516 (GRCh38/hg38)); and/or GeneHancer Identifier: GH02J156319 (i.e. chr2:156319401-156321392 (GRCh38/hg38)); a promoter region of a NR4A1 gene (Nuclear Receptor Subfamily 4 Group A Member 1)(e.g., a promoter as designated by GeneCard ID: GC12P052022; chr12:52,022,832-52,059,507 (GRCh38/hg38); GeneHancer Identifier: GH12J052049 (i.e. chr12:52049310-52065715 (GRCh38/hg38)); GeneHancer Identifier: GH12J052041 (i.e. chr12:52041026-52047831 (GRCh38/hg38)); and/or GeneHancer Identifier: GH12J052023 (i.e. chr12:52021200-52022401 (GRCh38/hg38)); a promoter region of a CSRNP2 gene (Cysteine And Serine Rich Nuclear Protein 2)(e.g., a promoter as designated by GeneCard ID: GC12M051061; chr12:51,061,205-51,083,664 (GRCh38/hg38); GeneHancer Identifier: GH12J051081; GeneHancer Identifier: GH12J051084; GeneHancer Identifier: GH12J051019 GeneHancer Identifier: GH12J050399; or GeneHancer Identifier: GH12J052068); a promoter region of a PHLDA1 gene (Pleckstrin Homology Like Domain Family A Member 1)(e.g., a promoter as designated by GeneCard ID: GC12M076025; chr12:76,025,447-76,031,776 (GRCh38/hg38); GeneHancer Identifier: GH12J076017; GeneHancer Identifier: GH12J075974; GeneHancer Identifier: GH12J075508; GeneHancer Identifier: GH12J076080; and/or GeneHancer Identifier: GH12J075819); a promoter region of a GAN gene (Gigaxonin)(e.g., a promoter as designated by GeneCard ID: GC16P081319; chr16:81,314,944-81,390,809 (GRCh38/hg38); GeneHancer Identifier: GH16J081312; GeneHancer Identifier: GH16J081731; GeneHancer Identifier: GH16J081813; and/or GeneHancer Identifier: GH16J081745); a promoter region of a SLC25A25 gene (Solute Carrier Family 25 Member 25)(e.g., a promoter as designated by GeneCard ID: GC09P128068; chr9:128,068,200-128,109,245 (GRCh38/hg38); GeneHancer Identifier: GH09J128090; GeneHancer Identifier: GH09J128065; GeneHancer Identifier: GH09J127962; and/or GeneHancer Identifier: GH09J127761); a promoter region of a SLC35E4 gene (Solute Carrier Family 35 Member E4)(e.g., a promoter as designated by GeneCard ID: GC22P032585; chr22:30,634, 148-30,669,019 (GRCh38/hg38); GeneHancer Identifier: GH22J030634; GeneHancer Identifier: GH22J030666; GeneHancer Identifier: GH22J030572; and/or GeneHancer Identifier: GH22J030589); the promoter region of a CAND2 gene (Cullin Associated And Neddylation Dissociated 2)(e.g., a promoter as designated by GeneCard ID: GC03P012814; chr3:12,796,472-12,871,916 (GRCh38/hg38); GeneHancer Identifier: GH03J012795; and/or GeneHancer Identifier: GH03J012757); a promoter region of a SNCAIP gene (Synuclein Alpha Interacting Protein)(e.g., a promoter as designated by GeneCard ID: GC05P122311; chr5:122,311,354-122,464,219 (GRCh38/hg38); and/or GeneHancer Identifier: GH05J122311); a promoter region of a WNT9A gene (Wnt Family Member 9A)(e.g., a promoter as designated by GeneCard ID: GC01M227920; chr1:227,918,656-227,947,932 (GRCh38/hg38); GeneHancer Identifier: GH01J227942; and/or GeneHancer Identifier: GH01J227891); a promoter region of a EGR1 gene (Early Growth Response 1)(e.g., a promoter as designated by GeneCard ID: GC05P138465; chr5:138,465,479-138,469,303 (GRCh38/hg38); GeneHancer Identifier: GH05J138463; and/or GeneHancer Identifier: GH05J138490); a promoter region of a DUSP2 gene (Dual Specificity Phosphatase 2)(e.g., a promoter as designated by GeneCard ID: GC02M097442; chr2:96, 143, 166-96, 145,468 (GRCh38/hg38); GeneHancer Identifier: GH02J096142; GeneHancer Identifier: GH02J096255; GeneHancer Identifier: GH02J096304; GeneHancer Identifier: GH02J096263; and/or GeneHancer Identifier: GH02J096009); and/or a promoter region of a SPRY4 gene (Sprouty RTK Signaling Antagonist 4)(e.g., a promoter as designated by GeneCard ID: GC05M142310; chr5:142,310,427-142,326,455 (GRCh38/hg38); GeneHancer Identifier: GH05J142314; or GeneHancer Identifier: GH05J142355); and/or a promoter region of a NFKB gene. In one embodiment, the promoter region is a NFKB circuit having five consensus sequences approximating the NF-κB canonical recognition motif based on genes up-regulated through inflammatory challenge: InfB1, 116, Mcp1, Adamts5, and Cxcl10.

In each of the above embodiments, a promoter sequence may be an ortholog, paralog, or homolog of anyone of the above referenced promoters. In one aspect, the transcriptional regulatory region is a fragment of any of the above promoter sequences. In one embodiment, the transcriptional regulatory region is a promoter nucleic acid sequence from two or more distinct mechanically-responsive genes. In one embodiment, when the cellular sensor is TRPV4, the transcriptional regulatory region of the synthetic circuit is selected from a nucleic acid sequence corresponding to a promoter derived from NF-κB, PTGS2, FGF1, PMEPA1, IL11, LAMC2, LAMA3, HBEGF, JunB, ATF3, INHBA, CCN1, NGF, TGFβ1, ERF, FOS, KLF4, TEAD4, TNFRSF11B, and any combination thereof. In another aspect, when the cellular sensor is PIEZO1, the transcriptional regulatory region of the synthetic circuit is selected from a nucleic acid sequence corresponding to a promoter from ADMTS1, NR4A1, NR4A2, WNT9A, SPRY4 and any combination thereof.

In addition, the transcriptional regulatory region from a mechanically-responsive gene (and combinations thereof) can be selected based on the desired temporal delivery of the biologic therapeutic. For example, the disclosure provides mechanical loading activates NFKBr-IL1Ra tissues by 24 hours, as measured by an increase in IL-1Ra produced by loaded tissue constructs and resumed baseline activity levels after 24 hours. Conversely, mechanical loading activates PTGS2-IL1Ra in the 24 hours after loading and found that tissues produced more IL-1Ra after 48 hours. Thus, a synthetic circuit according to the present disclosure provides the use of distinct signaling networks for defining the specificity, timing, and dose response for the expression of therapeutic biologic drugs. Using an engineered, living tissue construct for coordinated drug delivery obviates many of the traditional limitations of “smart” materials, such as long-term integration, rapid dynamic responses, and extended drug delivery without the need for replacement or reimplantation of the drug delivery system. The modular approach of using cells as both the mechanosensors and the effectors within engineered tissues allows regulation and sensitivity at the mechanically sensitive channel or receptor level, the signal network level, and the gene circuit level.

In some embodiments, the transcriptional regulatory region is placed upstream of a minimal promoter element and a nucleic acid sequence encoding the therapeutic biologic. For example, a TATA box derived from, in a non-limiting example, the minimal CMV promoter can be placed between the transcriptional regulatory region and therapeutic biologic such that binding of one or more transcription factors to the transcriptional regulatory region leads to transcription of the therapeutic biologic. In each of the above embodiments, additional regulatory elements can be included in the synthetic circuit as using common knowledge and methods in the art.

In one embodiment, the therapeutic biologic may include in non-limiting examples any range of endogenous or exogenous (e.g., non-mammalian) genes with therapeutic or diagnostic applications, including anti-catabolic, anti-inflammatory, pro-anabolic, pro-regenerative, pro-circadian, anti-microbial, anti-pain outputs, morphogens, growth factors, anti-cancer, etc. The proper therapeutic biologic will vary depending upon the host treated and the particular condition, disease, or disorder to be treated. In an exemplary embodiment, the therapeutic biologic is the cytokine antagonists IL-1 receptor antagonist (IL-1ra) or the type I soluble TNF receptor (sTNFR1). In some embodiments, the therapeutic biologic can be, miRNA, a shRNA, a therapeutic growth factor, a transcriptional regulator, an extracellular matrix (ECM) protein, an anti-inflammatory protein, or a biomarker used to monitor treatment efficacy or disease progression. For example, the transgene may be sTNFR1, IL-1Ra, IL6, IkB-alph, IL-10, a suicide gene, or a matrix degrading enzyme, such as a matrix metalloproteinase (MMP).

In one aspect, when the desired therapeutic is anti-inflammatory the therapeutic biologic nucleic acid sequence encodes for one or more of IL-1Ra, sTNFR1/2, IL-10 and IL-4. In another aspect, when the desired therapeutic response is pro-anabolic, the therapeutic biologic nucleic acid sequence encodes for one or more of a growth factor from the TGFβ superfamily, IGF, CTGF, FGF, and PDGF. In another aspect, when the desired therapeutic response is anti-pain, the therapeutic biologic nucleic acid sequence encodes for one or more of a kappa opioid ligand pro-peptide (prodynorphin), mu/delta opioid ligand pro-peptide (proenkephalin), delta/mu opioid ligand pro-peptide (proopiomelanocortin), and endocannabinoid ligand synthesis drivers. In another aspect, when the desired therapeutic response is anti-cancer, the therapeutic biologic nucleic acid sequence encodes for one or more of a TNF, IL-7, IL-15, IL-12, IL-2, IFN (based on tumor mechanobiology). In another embodiment, when the target cell is a smooth muscle cell, the therapeutic biologic nucleic acid molecule encodes for one or more of NOS and PTGIS. In still another embodiment, when the desired therapeutic response is anti-fibrotic, the therapeutic biologic molecule encodes one or more of Decorin, TGFβ-receptor, MMPs, ALDH2 and NR3C1.

The present disclosure provides vectors and constructs for producing genetically modified cells comprising the synthetic circuit of the disclosure. A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A constructs of the present disclosure can contain a synthetic circuit of the disclosure operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and preferably, is a DNA plasmid.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

In some embodiments genetically modified cells of the disclosure may be generated by cloning a nucleic acid sequence comprising a synthetic circuit of the disclosure into a viral vector. Thus, genetic modification of a target cell can be accomplished by transducing a substantially homogeneous cell composition with a recombinant DNA construct. In certain embodiments, a retroviral vector (either gamma-retroviral or lentiviral) is employed for the introduction of the DNA construct into the cell. For example, a polynucleotide comprising the regulatory sequences of the disclosure and/or a nucleic acid sequence encoding a therapeutic biologic can be cloned into a retroviral vector and expression can be driven from its endogenous promoter, from the retroviral long terminal repeat, or from a promoter specific for a target cell type of interest. Other viral vectors, or non-viral vectors may be used as well.

For initial genetic modification of a target cell, a retroviral vector is generally employed for transduction, however any other suitable viral vector or non-viral delivery system can be used. The nucleic acid sequences can be constructed with an auxiliary molecule (e.g., a cytokine) in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). In certain embodiments, any vector disclosed herein can comprise a P2A peptide. Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Nat. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J Clin. Invest. 89:1817.

Other transducing viral vectors can be used to genetically modify a target cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

Non-viral approaches can also be employed for genetic modification of a target cell. For example, a nucleic acid molecule can be introduced into a target cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue, are injected systemically, or incorporated into an implantable tissue which is the administered to a subject. Recombinant receptors can also be derived or obtained using transposases or targeted nucleases (e.g. Zinc finger nucleases, meganucleases, or TALE nucleases, CRISPR). Transient expression may be obtained by RNA electroporation.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to forma single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome.cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

Any targeted genome editing methods can be used to place presently disclosed synthetic circuit at one or more endogenous gene loci of a presently disclosed target cell. In certain embodiments, a CRISPR system is used to deliver presently disclosed synthetic circuit to one or more endogenous gene loci of a presently disclosed target cell. In certain embodiments, zinc-finger nucleases are used to deliver presently disclosed synthetic circuit to one or more endogenous gene loci of a presently disclosed target cell. In certain embodiments, a TALEN system is used to deliver presently disclosed synthetic circuit to one or more endogenous gene loci of a presently disclosed target cell.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

Placement of a presently disclosed synthetic circuit of the disclosure can be made at any endogenous gene locus.

### b) Genetically Modified Cells

The target cells that are used to generate the genetically modified cells may be in non-limiting examples stem cells, such as embryonic stem cells (ES) and adult stem cells (somatic stem cells or tissue-specific stem cells), induced pluripotent stem cells (iPSCs), progenitor cells, fibroblasts, cardiomyocytes, hepatocytes, endothelial cells, chondrocytes, smooth or striated muscle cells, bone cells, synovial cells, tendon cells, ligament cells, meniscus cells, adipose cells, splenocytes, epithelial cells, neurons, astrocytes, microglial cells, vascular cells, B-cells, dendritic cells, natural killer cells, or T-cells. Target cells can be derived and maintained using common methods known in the art.

“Stem cells” as used herein refers to an undifferentiated cell of a multicellular organism that is capable of giving rise to indefinitely more cells of the same type, and from which certain other kinds of cell arise by differentiation. Stem cells can differentiate into specialized cells and can divide (through mitosis) to produce more stem cells. They are found in multicellular organisms. In mammals, there are two broad types of stem cells: embryonic stem cells, which are isolated from the inner cell mass of blastocysts, and adult stem cells, which are found in various tissues. In adult organisms, stem cells and progenitor cells act as a repair system for the body, replenishing adult tissues. In a developing embryo, stem cells can differentiate into all the specialized cells, such as ectoderm, endoderm and mesoderm, but also maintain the normal turnover of regenerative organs, such as blood, skin, or intestinal tissues.

“Induced pluripotent stem cells” or “iPSCs” as used interchangeably herein refers to a type of pluripotent stem cell that can be artificially derived from a non-pluripotent cell, typically an adult somatic cell, by inducing a “forced” expression of certain genes and transcription factors.

A “progenitor cell” as used herein refers to a biological cell that, like a stem cell, has a tendency to differentiate into a specific type of cell, but is already more specific than a stem cell. While stem cells can replicate indefinitely, progenitor cells can divide only a limited number of times.

(ES) cells are isolated from the inner cell mass of blastocysts of preimplantation-stage embryos. These cells require specific signals to differentiate to the desired cell type; if simply injected directly, they will differentiate into many different types of cells, resulting in a tumor derived from this abnormal pluripotent cell development (a teratoma). The directed differentiation of ES cells and avoidance of transplant rejection are just two of the hurdles that ES cell researchers still face. With their potential for unlimited expansion and pluripotency, ES cells are a potential source for regenerative medicine and tissue replacement after injury or disease.

Induced stem cells (iSC) are stem cells artificially derived from somatic, reproductive, pluripotent or other cell types by deliberate epigenetic reprogramming. They are classified as totipotent (iTC), pluripotent (iPSC) or progenitor (multipotent-iMSC, also called an induced multipotent progenitor cell-iMPC) or unipotent (iUSC) according to their developmental potential and degree of dedifferentiation.

iPSCs are somatic cells that have been genetically reprogrammed to an embryonic stem cell-like state by being forced to express genes important for maintaining the defining properties of embryonic stem cells. Although additional research is needed, iPSCs are already useful tools for drug development and modeling of diseases, and scientists hope to use them in transplantation medicine. In addition, tissues derived from iPSCs will be a nearly identical match to the cell donor and thus probably avoid rejection by the immune system. By studying iPSCs and other types of pluripotent stem cells, researchers may learn how to reprogram cells to repair damaged tissues in the human body.

Adult stem cells are undifferentiated cells, found throughout the body after development that multiply by cell division to replenish dying cells and regenerate damaged tissues. Also known as somatic stem cells, they can be found in juvenile as well as adult animals and human bodies. Scientific interest in adult stem cells is centered on their ability to divide or self-renew indefinitely, and generate all the cell types of the organ from which they originate, potentially regenerating the entire organ from a few cells. Unlike embryonic stem cells, the use of human adult stem cells in research and therapy is not considered to be controversial, as they are derived from adult tissue samples rather than human 5 day old embryos generated by IVF (in vitro fertility) clinics designated for scientific research. They have mainly been studied in humans and model organisms such as mice and rat.

The production of adult stem cells does not require the destruction of an embryo. Additionally, when adult stem cells are obtained from the intended recipient (an autograft) there is no risk of immune rejection. Adult stem cell treatments have been successfully used for many years to treat leukemia and related bone/blood cancers through bone marrow transplants.

Hematopoietic stem cells are found in the bone marrow and umbilical cord blood and give rise to all the blood cell types. Mesenchymal stem cells (MSCs) are of stromal origin and may differentiate into a variety of tissues and cell types, including: osteoblasts (bone cells), chondrocytes (cartilage cells), myocytes (muscle cells) adipocytes (fat cells). MSCs have been isolated from placenta, adipose tissue, lung, bone marrow and blood, Wharton's jelly from the umbilical cord and teeth (perivascular niche of dental pulp and periodontal ligament). MSCs are attractive for clinical therapy due to their ability to differentiate, provide trophic support, and modulate innate immune response.

Endothelial stem cells are one of the three types of multipotent stem cells found in the bone marrow. They are a rare and controversial group with the ability to differentiate into endothelial cells, the cells that line blood vessels.

Self-renewing tissues, such as the epidermis and hair follicle, continuously generate new cells to replenish the dead squames and hairs, which are sloughed into the environment. Therefore, perhaps the simplest definition of an epithelial stem cell is based on lineage: a stem cell is the cell of origin for terminally differentiated cells in adult tissues. For example, tracing the lineage of a corneocyte or hair cell back to its ultimate source in the adult skin leads to a stem cell. However, because the tools required to perform lineage analysis have not been available until recently, investigators have principally adopted definitions from the hematopoietic system. In particular, stem cells were felt to be self-renewing, multipotent, and clonogenic, similar to stem cells in the hematopoietic system that can regenerate all of the blood lineages from one cell after transplantation. In contrast to the hematopoietic stem cell field, cutaneous epithelial stem cell biologists also relied heavily on quiescence as a major stem cell characteristic.

The existence of stem cells in the adult brain has been postulated following the discovery that the process of neurogenesis, the birth of new neurons, continues into adulthood in rats. The presence of stem cells in the mature primate brain was first reported in 1967. It has since been shown that new neurons arc generated in adult mice, songbirds and primates, including humans. Normally, adult neurogenesis is restricted to two areas of the brain—the subventricular zone, which lines the lateral ventricles, and the dentate gyrus of the hippocampal formation. Although the generation of new neurons in the hippocampus is well established, the presence of true self-renewing stem cells there has been debated. Under certain circumstances, such as following tissue damage in ischemia, neurogenesis can be induced in other brain regions, including the neocortex. Neural stem cells are commonly cultured in vitro as so called neurospheres—floating heterogeneous aggregates of cells, containing a large proportion of stem cells. They can be propagated for extended periods of time and differentiated into both neuronal and glia cells, and therefore behave as stem cells. However, some recent studies suggest that this behavior is induced by the culture conditions in progenitor cells, the progeny of stem cell division that normally undergo a strictly limited number of replication cycles in vivo. Furthermore, neurosphere-derived cells do not behave as stem cells when transplanted back into the brain.

Neural stem cells share many properties with hematopoietic stem cells (HSCs). Remarkably, when injected into the blood, neurosphere-derived cells differentiate into various cell types of the immune system.

Mammary stem cells provide the source of cells for growth of the mammary gland during puberty and gestation and play an important role in carcinogenesis of the breast. Mammary stem cells have been isolated from human and mouse tissue as well as from cell lines derived from the mammary gland. Single such cells can give rise to both the luminal and myoepithelial cell types of the gland, and have been shown to have the ability to regenerate the entire organ in mice.

Intestinal stem cells divide continuously throughout life and use a complex genetic program to produce the cells lining the surface of the small and large intestines. Intestinal stem cells reside near the base of the stem cell niche, called the crypts of Lieberkuhn. Intestinal stem cells are probably the source of most cancers of the small intestine and colon.

Olfactory adult stem cells have been successfully harvested from the human olfactory mucosa cells, which are found in the lining of the nose and are involved in the sense of smell. If they are given the right chemical environment these cells have the same ability as embryonic stem cells to develop into many different cell types. Olfactory stem cells hold the potential for therapeutic applications and, in contrast to neural stem cells, can be harvested with case without harm to the patient. This means they can be easily obtained from all individuals, including older patients who might be most in need of stem cell therapies.

Hair follicles contain two types of stem cells, one of which appears to represent a remnant of the stem cells of the embryonic neural crest. Similar cells have been found in the gastrointestinal tract, sciatic nerve, cardiac outflow tract and spinal and sympathetic ganglia. These cells can generate neurons, Schwann cells, myofibroblast, chondrocytes and melanocytes.

Multipotent stem cells with a claimed equivalency to embryonic stem cells have been derived from spermatogonial progenitor cells found in the testicles of laboratory mice. The extracted stem cells are known as human adult germline biggmacc stem cells (GSCs). Multipotent stem cells have also been derived from germ cells found in human testicles.

Induced stem cells (iSC) are stem cells artificially derived from somatic, reproductive, pluripotent or other cell types by deliberate epigenetic reprogramming. They are classified as totipotent (iTC), pluripotent (iPSC) or progenitor (multipotent-iMSC, also called an induced multipotent progenitor cell-iMPC) or unipotent (iUSC) according to their developmental potential and degree of dedifferentiation.

iPSCs are somatic cells that have been genetically reprogrammed to an embryonic stem cell-like state by being forced to express genes important for maintaining the defining properties of embryonic stem cells. Although additional research is needed, iPSCs are already useful tools for drug development and modeling of diseases, and scientists hope to use them in transplantation medicine. In addition, tissues derived from iPSCs will be a nearly identical match to the cell donor and thus probably avoid rejection by the immune system. By studying iPSCs and other types of pluripotent stem cells, researchers may learn how to reprogram cells to repair damaged tissues in the human body.

Chronic lung diseases such as idiopathic pulmonary fibrosis and cystic fibrosis or chronic obstructive pulmonary disease and asthma are leading causes of morbidity and mortality worldwide with a considerable human, societal, and financial burden. Several protocols have been developed for generation of the most cell types of the respiratory system, which may be useful for deriving patient-specific therapeutic cells.

Some lines of iPSCs have the potentiality to differentiate into male germ cells and oocyte-like cells in an appropriate niche (by culturing in retinoic acid and porcine follicular fluid differentiation medium or seminiferous tubule transplantation). Moreover, iPSC transplantation make a contribution to repairing the testis of infertile mice, demonstrating the potentiality of gamete derivation from iPSCs in vivo and in vitro.

T cells are a type of lymphocyte (in turn, a type of white blood cell) that play a central role in cell-mediated immunity. They can be distinguished from other lymphocytes, such as B cells and natural killer cells (NK cells), by the presence of a T-cell receptor (TCR) on the cell surface. They are called T cells because they mature in the thymus (although some also mature in the tonsils). The several subsets of T cells each have a distinct function. The majority of human T cells rearranges their alpha/beta T cell receptors and are termed alpha beta T cells and are part of adaptive immune system. Specialized gamma delta T cells, which comprise a minority of T cells in the human body (more frequent in ruminants), have invariant TCR (with limited diversity), can effectively present antigens to other T cells and are considered to be part of the innate immune system.

The T cell includes any of a CD8-positive T cell (cytotoxic T cell: CTL), a CD4-positive T cell (helper T cell), a suppressor T cell, a regulatory T cell such as a controlling T cell, an effector cell, a naive T cell, a memory T cell, an al3T cell expressing TCR a and P chains, and a y6T cell expressing TCR y and 6 chains. The T cell includes a precursor cell of a T cell in which differentiation into a T cell is directed. Examples of “cell populations containing T cells” include, in addition to body fluids such as blood (peripheral blood, umbilical blood etc.) and bone marrow fluids, cell populations containing peripheral blood mononuclear cells (PBMC), hematopoietic cells, hematopoietic stem cells, umbilical blood mononuclear cells etc., which have been collected, isolated, purified or induced from the body fluids. Further, a variety of cell populations containing T cells and derived from hematopoietic cells can be used in the present invention. These cells may have been activated by cytokine such as 1L-2 in vivo or ex vivo. As these cells, any of cells collected from a living body, or cells obtained via ex vivo culture, for example, a T cell population obtained by the method of the present invention as it is, or obtained by freeze preservation, can be used.

Artificial T cell receptors (also known as chimeric T cell receptors, chimeric immunoreceptors, chimeric antigen receptors (CARs)) are engineered receptors, which graft an arbitrary specificity onto an immune effector cell. These receptors may be used to graft the specificity of a monoclonal antibody onto a T cell. CARs may consist of a monoclonal antibody fragment, such as a single-chain variable fragment (scFv), that presents on the outside of T-cell membranes, and is fused to intraceullarly-facing stimulatory molecules. The scFv portion may recognize the tumor target. Upon binding, the intracellular stimulatory portions may initiate a signal to activate the T cell.

Artificial T cell receptors may be used as therapy for cancer using adoptive cell transfer. T cells are removed from a patient and modified so that they express receptors specific to the particular form of cancer. The T cells, which can then recognize and kill the cancer cells, are reintroduced into the patient. Modification of T-cells sourced from donors other than the patient may also be used.

The present disclosure also provides pharmaceutical compositions. The pharmaceutical composition comprises a plurality of genetically modified cells according to the present disclosure, as an active component, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

Compositions comprising the presently disclosed genetically modified cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The quantity of cells to be administered will vary for the subject being treated. In a one embodiment, between about 10³ and about 10¹⁰, between about 10⁵ and about 10⁹, or between about 10⁶ and about 10⁸ of the presently disclosed genetically modified cells are administered to a human subject. More effective cells may be administered in even smaller numbers. In certain embodiments, at least about 1×10⁸, about 2×10⁸, about 3×10⁸, about 4×10⁸, or about 5×10⁸ of the presently disclosed genetically modified cells are administered to a human subject. In certain embodiments, between about 1×10⁷ and 5×10⁸ of the presently disclosed genetically modified cells are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD₅₀ in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

Compositions comprising the presently disclosed genetically modified cells can be provided systemically or locally to a subject for inducing and/or enhancing a therapeutic response to a condition, disease, or disorder. In certain embodiments, the presently disclosed genetically modified cells or compositions comprising the same are directly injected into a tissue or organ of interest (e.g., an organ affected by a condition, disease or disorder). Alternatively, the presently disclosed genetically modified cells or compositions comprising the same are provided indirectly to the organ of interest, for example, by administration into the circulatory system (e.g., the organ vasculature). Expansion and differentiation agents can be provided prior to, during or after administration of the cells in vitro or in vivo.

The presently disclosed genetically modified cells can be administered in any physiologically acceptable vehicle, normally intravascularly, although they may also be introduced into bone or other convenient site where the cells may find an appropriate site for regeneration and differentiation (e.g., lymphatics). Usually, at least a population of about 1×10⁵ cells will be administered. The presently disclosed genetically modified cells can comprise a purified population of cells. Those skilled in the art can readily determine the percentage of the presently genetically modified cells in a population using various well-known methods, such as fluorescence activated cell sorting (FACS). Suitable ranges of purity in populations comprising the presently disclosed genetically modified cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The presently disclosed compositions can be pharmaceutical compositions comprising the presently disclosed genetically modified cells and a pharmaceutically acceptable carrier. Administration can be autologous or heterologous. For example, target cells can be obtained from one subject, genetically modified according to the disclosure and administered to the same subject or a different, compatible subject. Peripheral blood derived genetically modified cells (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed genetically modified cells), it can be formulated in a unit dosage injectable form (solution, suspension, emulsion).

### II. Methods

Cells disclosed herein, and/or generated using the methods disclosed herein, may be used in cellular therapy and adoptive cell transfer, for the treatment, or the manufacture of a medicament for treatment, of a variety of disorders, diseases (e.g., autoimmune diseases, inflammatory diseases), and other conditions (e.g., osteoporosis).

Thus, aspects of the present disclosure is a method for treating a subject in need thereof. The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented.

Also provided is a process of treating or preventing an inflammatory or proliferative disease, disorder, or condition in a subject in need of by administration of a therapeutically effective amount of a genetically modified cells as described.

Methods described herein are generally performed on a subject in need thereof. A subject in need of the therapeutic methods described herein can be a subject having, diagnosed with, suspected of having, or at risk for developing a disease, disorder, or condition. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of genetically modified cells is, for example, that amount that would cause the desired therapeutic effect in a subject while minimizing undesired side effects. In various embodiments, an effective amount of genetically modified cells described herein can substantially inhibit progression of disease, disorder, or condition, slow the progress of disease, disorder, or condition, or limit the development of disease, disorder, or condition.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of the disclosed genetically modified cells can occur as a single event or over a time course of treatment. For example, genetically modified cells-based therapy can be administered daily, weekly, bi-weekly, or monthly. For more chronic conditions, treatment could extend from several weeks to several months or years.

Treatment in accord with the methods described herein can be performed prior to, concurrent with, or after conventional treatment modalities for the disease, disorder, or condition.

The genetically modified cell-therapy can be administered simultaneously or sequentially with another agent, such as an anti-inflammatory therapy, or another agent. For example, a genetically modified cell-therapy can be administered before, after, or simultaneously with another agent, such as a chemotherapeutic agent, another form of immune therapy, or radiation therapy. Simultaneous administration can occur through the administration of separate compositions, each containing one or more of a genetically modified cell-therapy and another agent, such as a chemotherapeutic agent, additional immune therapy, or radiation therapy. Simultaneous administration can occur through the administration of one composition containing two or more of genetically modified cell-therapy, an antibiotic, an anti-inflammatory, or another agent, such as a chemotherapeutic agent, immune therapy, or radiation therapy.

The administration of genetically modified cells or a population of genetically modified cells of the present disclosure be carried out by aerosol inhalation, injection, ingestion, transfusion, implantation or transplantation. The genetically modified cells, may be administered to a patient subcutaneously, intradermally, intratumorally, intranodally, intramedullary, intramuscularly, by intravenous or intralymphatic injection, or intraperitoneally. In one embodiment, the cell compositions of the present disclosure are preferably administered by intravenous injection.

As noted above, the administration of genetically modified cells or a population of genetically modified cells can consist of the administration of 10³-10⁹ cells per kg body weight, preferably 10⁵ to 10⁶ cells/kg body weight including all integer values of cell numbers within those ranges. The genetically modified cells or a population of genetically modified cells can be administrated in one or more doses. In another embodiment, the effective amount of genetically modified cells or a population of genetically modified cells are administrated as a single dose. In another embodiment, the effective amount of cells are administered as more than one dose over a period time. Timing of administration is within the judgment of a health care provider and depends on the clinical condition of the patient. The genetically modified cells or a population of genetically modified cells may be obtained from any source, such as a blood bank or a donor. While the needs of a patient vary, determination of optimal ranges of effective amounts of a given genetically modified cells population(s) for a particular disease or conditions are within the skill of the art. An effective amount means an amount which provides a therapeutic or prophylactic benefit. The dosage administered will be dependent upon the age, health and weight of the patient recipient, type of concurrent treatment, if any, frequency of treatment, and the nature of the effect desired.

The present disclosure is directed to a method of treating a subject in need thereof. The method comprises administering to subject the composition for cell therapy, as described above. The subject may have diseases include a variety of acute and chronic diseases including but not limited to genetic, degenerative, or autoimmune diseases and obesity related conditions. Diseases include acute and chronic immune and autoimmune pathologies, such as, but not limited to, rheumatoid arthritis (RA), juvenile chronic arthritis (JCA), tissue ischemia, thyroiditis, graft versus host disease (GVHD), scleroderma, diabetes mellitus, Graves' disease, disc degeneration and low back pain, allergy, acute or chronic immune disease associated with an allogenic transplantation, such as, but not limited to, renal transplantation, cardiac transplantation, bone marrow transplantation, liver transplantation, pancreatic transplantation, small intestine transplantation, lung transplantation and skin transplantation; infections, including, but not limited to, sepsis syndrome, cachexia, circulatory collapse and shock resulting from acute or chronic bacterial infection, acute and chronic parasitic and/or infectious diseases, bacterial, viral or fungal, such as a human immunodeficiency virus (HIV), acquired immunodeficiency syndrome (AIDS) (including symptoms of cachexia, autoimmune disorders, AIDS dementia complex and infections); inflammatory diseases, such as chronic inflammatory pathologies, including chronic inflammatory pathologies such as, but not limited to, sarcoidosis, chronic inflammatory bowel disease, ulcerative colitis, osteogenesis imperfecta, and Crohn's pathology or disease; vascular inflammatory pathologies, such as, but not limited to, disseminated intravascular coagulation, atherosclerosis, Kawasaki's pathology and vasculitis syndromes, such as, but not limited to, polyarteritis nodosa, Wegener's granulomatosis, Henoch-Schonlein purpura, giant cell arthritis and microscopic vasculitis of the kidneys; chronic active hepatitis; Sjogren's syndrome; spondyloarthropathies, such as ankylosing spondylitis, psoriatic arthritis and spondylitis, osteopathic arthritis and spondylitis, reactive arthritis and arthritis associated with inflammatory bowel disease; and uveitis; neurodegenerative diseases, including, but not limited to, demyelinating diseases, such as multiple sclerosis and acute transverse myelitis; myasthenia gravis; extrapyramidal and cerebellar disorders, such as lesions of the corticospinal system; disorders of the basal ganglia or cerebellar disorders; hyperkinetic movement disorders, such as Huntington's chorea and senile chorea; drug-induced movement disorders, such as those induced by drugs which block central nervous system (CNS) dopamine receptors; hypokinetic movement disorders, such as Parkinson's disease; progressive supranuclear palsy; cerebellar and spinocerebellar disorders, such as a structural lesions of the cerebellum; spinocerebellar degenerations (spinal ataxia, Friedreich's ataxia, cerebellar cortical degenerations, multiple systems degenerations (Mencel, Dejerine-Thomas, Shi-Drager, and MachadoJoseph)); and systemic disorders (Refsum's disease, abetalipoprotienemia, ataxia, telangiectasia, and mitochondrial multisystem disorder); disorders of the motor unit, such as neurogenic muscular atrophies (anterior horn cell degeneration, such as amyotrophic lateral sclerosis, infantile spinal muscular atrophy and juvenile spinal muscular atrophy); Alzheimer's disease; Down's syndrome in middle age; diffuse Lewy body disease; senile dementia of Lewy body type; Wernicke-Korsakoff syndrome; chronic alcoholism; primary biliary cirrhosis; cryptogenic fibrosing alveolitis and other fibrotic lung diseases; hemolytic anemia; Creutzfeldt-Jakob disease; subacute sclerosing panencephalitis, Hallervorden-Spatz disease; and dementia pugilistica, or any subset thereof; and malignant pathologies involving TNF-secreting tumors or other malignancies involving TNF, such as, but not limited to, leukemias (acute, chronic myelocytic, chronic lymphocytic and/or myelodyspastic syndrome); lymphomas (Hodgkin's and non-Hodgkin's lymphomas, such as malignant lymphomas (Burkitt's lymphoma or Mycosis fungoidcs)).

### a) Chronic Inflammatory Disease

Chronic inflammatory diseases such as arthritis are characterized by aberrant activity of cytokines such as tumor necrosis factor-α (TNF-α) and interleukin-1 (IL-1). These pro-inflammatory mediators are expressed by a wide variety of cells in musculoskeletal tissues, including myotubes, satellite cells, chondrocytes, synovial fibroblasts, osteoblasts, and resident as well as infiltrating innate immune cells. These cell types are also capable of responding to TNF-α and IL-1 a through canonical signaling via their cognate cell surface receptors. In healthy tissue, appropriate signaling of TNF-α and IL-1 contributes to organ and tissue homeostasis. In this state, these mediators promote tissue remodeling, orchestrate phagocytosis of cellular debris and immunogenic substrates, and coordinate transitions between niche stem cell quiescence and proliferation/differentiation programs. TNF-α has also been shown to enhance stem cell differentiation in a variety of efforts to enhance MSC osteogenesis. However, in chronic diseases, elevated levels of these pro-inflammatory cytokines can lead directly to pain, cytotoxicity, accelerated tissue catabolismor wasting, and exhaustion of resident stem cell niches.

A regenerative medicine approach may be used to treat chronic inflammatory diseases by generating custom-designed cells that can execute real-time, programmed responses to environmental cues, including mechanically input. Modified cells, such as stem cells, may be generated with the ability to antagonize IL-1- and TNFa-mediated inflammation in a non-limiting example. Genome-edited stem cells may be used to engineer articular cartilage tissue to establish the efficacy of therapy toward protection of tissues against cytokine-induced degeneration. This approach of repurposing mechanotransduction signaling pathways may facilitate transient production of cytokine antagonists and permit effective treatment of chronic diseases while overcoming limitations associated with delivery of large drug doses or constitutive overexpression of biologic compounds.

A therapeutic molecule may be any number of exogenous anti-cytokine therapies that effectively counteract the negative sequelae of TNF-α and IL-1 dysregulation. For example, therapeutic molecules may include competitive antagonists such as IL-1 receptor antagonist (IL-1Ra, anakinra), which alleviate symptoms of rheumatoid arthritis and the onset of post-traumatic arthritis; anti-TNF therapies, such as the soluble type 2 TNF receptor (etanercept) and monoclonal antibodies to TNF-α (adalimumab, infliximab), which have demonstrated efficacy toward offsetting pain associated with chronic and rheumatic diseases, including arthritis, ankylosing spondylitis, Crohn disease, plaque psoriasis, and ulcerative colitis; type I soluble TNFR receptor (sTNFR1), which generally provided in the context of relatively high or unregulated doses.

### b) Cancer Therapy

The compositions may be used in methods of cancer therapy where the immune system is used to treat cancer. Immunotherapies fall into three main groups: cellular, antibody and cytokine. They exploit the fact that cancer cells often have subtly different molecules on their surface that can be detected by the immune system. These molecules, known as cancer antigens, are most commonly proteins, but also include molecules such as carbohydrates. Immunotherapy is used to provoke the immune system into attacking the tumor cells by using these antigens as targets.

The compositions may be used in cellular therapies, also known as cancer vaccines, usually involve the removal of immune cells from the blood or from a tumor. Immune cells specific for the tumor may be modified, cultured and returned to the patient where the immune cells attack the cancer. Cell types that can be used in this way are natural killer cells, lymphokine-activated killer cells, cytotoxic T cells and dendritic cells.

Interleukin-2 and interferon-a are examples of cytokines, proteins that regulate and coordinate the behavior of the immune system. They have the ability to enhance anti-tumor activity and thus can be used as cancer treatments. Interferon-a is used in the treatment of hairy-cell leukemia, AIDS-related Kaposi's sarcoma, follicular lymphoma, chronic myeloid leukemia and malignant melanoma. Interleukin-2 is used in the treatment of malignant melanoma and renal cell carcinoma.

Dendritic cell therapy provokes anti-tumor responses by causing dendritic cells to present tumor antigens. Dendritic cells present antigens to lymphocytes, which activates them, priming them to kill other cells that present the antigen. In cancer treatment they aid cancer antigen targeting. One method of inducing dendritic cells to present tumor antigens is by vaccination with short peptides (small parts of protein that correspond to the protein antigens on cancer cells). These peptides on their own do not stimulate a strong immune response and may be given in combination with adjuvants (highly immunogenic substances). This provokes a strong response, while also producing a (sometimes) robust anti-tumor response by the immune system. Other adjuvants include proteins or other chemicals that attract and/or activate dendritic cells, such as granulocyte macrophage colony-stimulating factor (GM-CSF). Dendritic cells can also be activated within the body (in vivo) by making tumor cells express (GM-CSF). This can be achieved by either genetically engineering tumor cells that produce GM-CSF or by infecting tumor cells with an oncolytic virus that expresses GM-CSF.

Another strategy is to remove dendritic cells from the blood of a patient and activate them outside the body (ex vivo). The dendritic cells are activated in the presence of tumor antigens, which may be a single tumor-specific peptide/protein or a tumor cell lysate (a solution of broken down tumor cells). These activated dendritic cells are put back into the body where they provoke an immune response to the cancer cells. Adjuvants are sometimes used systemically to increase the anti-tumor response provided by ex vivo activated dendritic cells. More modern dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as targets by antibodies to produce immune responses.

Cytokines are a broad group of proteins produced by many types of cells present within a tumor. They have the ability to modulate immune responses. The tumor often employs it to allow it to grow and manipulate the immune response. These immune-modulating effects allow them to be used as drugs to provoke an immune response. Two commonly used groups of cytokines are interferons and interleukins.

Interferons are cytokines produced by the immune system. They are usually involved in anti-viral response, but also have use for cancer. The three groups of interferons (IFNs) are type I (IFNa and IFN(3), type II (IFNy) and type III (IFNX). IFNa has been approved for use in hairy-cell leukemia, AIDS-related Kaposi's sarcoma, follicular lymphoma, chronic myeloid leukemia and melanoma. Type I and II IFNs have been researched extensively and although both types promote anti-tumor immune system effects, only type I IFNs have been shown to be clinically effective. IFN2 shows promise for its anti-tumor effects in animal models.

Interleukins are a group of cytokines with a wide array of immune system effects. Interleukin-2 is used in the treatment of malignant melanoma and renal cell carcinoma. In normal physiology it promotes both effector T cells and T-regulatory cells, but its exact mechanism in the treatment of cancer is unknown.

### c) Regenerative Therapy Using Cell Therapy

Regenerative medicine provides the exciting potential for cell-based therapies to treat many diseases and restore damaged tissues using engineered cells for musculoskeletal applications. Modified cells derived from a myriad of adult tissues and differentiated down a lineage of choice may be tailored at the scale of the genome with application-dependent features. The compositions described here have broad applicability in regenerative medicine. For example, the ability to immobilize gene delivery vehicles capable of dictating cell fate and orchestrating ECM deposition may allow future investigators to control the spatial patterning of tissue development. This approach could indeed be applied toward engineering tissues comprised of multiple cell types and organized into regions of varied and distinct ECM constituents, a persistent challenge in the field of orthopaedic tissue engineering. Furthermore, diseases may be treated that involve complex interactions between multiple organ systems, those that drive deterioration of tissues that are not amenable to total replacement, or those in which discrimination between pathologic and healthy tissue/cells may be subtle or require real-time determination for safe and effective alleviation of disease. Such conditions may be most efficiently addressed by cells that can infiltrate, intelligently detect dysfunction, and deploy predefined therapeutic programs to resolve anomalous behavior of endogenous cells or ECM disorganization/degeneration. Employing these and other tools from synthetic biology together under the auspices of a functional cellular and tissue engineering paradigm, which aims to fully characterize and recapitulate features critical for successful cell/tissue replacement, will likely serve to advance the field of regenerative medicine toward the establishment of clinically effective therapies for a host of diseases.

In some embodiments, the site-specific nucleases may be used to generate functional deficiencies or complete knock-out of the proteins coded by the targeted genes in human iPSCs. Genetically modified iPSCs may be differentiated into chondrocytes using established techniques. Feedback-controlled gene circuits may be designed to modulate the production of soluble TNF receptors—specifically soluble TNF receptor 1 (sTNFR1), which blocks TNF signaling—in response to dynamic TNF levels. In some embodiments, this process may be performed in induced pluripotent stem cells (iPSCs), which can be expanded indefinitely, thus facilitating the complex genetic manipulations required for genome editing. To produce an implant with long-term in vivo stability, the rewired iPSCs may be differentiated into cartilage cells (chondrocytes), a robust, non-migratory cell that naturally responds to TNF. These cells may be formed into a tissue-engineered cartilage implant that can be implanted in the joint to repair damaged cartilage or subcutaneously to provide self-regulated, systemic anti-TNF.

### d) Osteoarthritis

The modified cells may be used in musculoskeletal regenerative medicine applications, such as developing therapies for osteoarthritis. Osteoarthritis (OA) is a progressive disease of synovial joints characterized by the destruction of articular cartilage. Surgical treatment options for focal cartilage defects include arthroscopic debridement, marrow stimulation via microfracture, and autologous transplantation of host tissue or ex vivo expanded autologous chondrocytes. Most of these surgical options lead to the development of a fibrocartilage matrix that serves only as a temporary solution to a complex and demanding biomechanical problem. For larger defects, joint arthroplasty serves as the most promising treatment option. While effective at restoring function to the joint, the need to revise an increasing number of primary arthroplasties means that a more functional, long-term solution is needed.

Inflammation plays a key role in the pathogenesis and progression of osteoarthritis (OA) and may compromise engineered tissue substitutes. Chondrocytes and synovial fibroblasts in OA joints are subjected to increased interleukin (IL)-1, IL-6, IL-17 and tumor necrosis factor (TNF)-a signaling. The activity of these cytokines in OA joints leads to increased production of matrix metalloproteinases (MMPs), aggrecanases, inducible nitric oxide synthase, and prostaglandin E2. These and other factors ultimately lead to suppression of cartilage-specific genes such as COL2A1, downregulation of proteoglycan levels, degeneration of extracellular matrix, and chondrocyte apoptosis. Furthermore, prolonged inflammatory signaling mediated by IL-la inhibits chondrogenic induction of stem cells and results in degradation of stem cell derived cartilage. The pro-inflammatory environment of the OA joint therefore necessitates a tissue substitute designed to resist inflammation-mediated degradation.

### e) Pain

Pain is generally defined as an unpleasant sensory and emotional experience associated with actual or potential tissue damage or described in terms of such damage. Merskey H, Bogduk N, eds., Classification of Chronic Pain, International Association for the Study of Pain (IASP) Task Force on Taxonomy, IASP Press: Seattle, 209-214, 1994. Because the perception of pain is highly subjective, it is one of the most difficult pathologies to diagnose and treat effectively.

In one aspect, provided herein is a method of treating an individual having pain, comprising administering to the individual a therapeutically effective amount of a composition comprising a genetically modified cell of the disclosure, wherein the therapeutically effective amount is an amount sufficient to cause a detectable improvement in said pain or a symptom associated with said pain. In one embodiment, said method additionally comprises determining a first level of pain in said individual prior to administration of said genetically modified cells, and determining a second level of pain in said individual after administration of said genetically modified cells, wherein said therapeutically effective amount of genetically modified cells reduces said second level of said pain as compared to said first level of pain.

In certain embodiments, the therapeutically effective amount of genetically modified cells, when administered, results in greater, or more long-lasting, improvement of pain in the individual as compared to administration of a placebo.

In certain embodiments, the pain is nociceptive pain. Nociceptive pain is typically elicited when noxious stimuli such as inflammatory chemical mediators are released following tissue injury, disease, or inflammation and are detected by normally functioning sensory receptors (nociceptors) at the site of injury. See, e.g., Koltzenburg, M. Clin. J. of Pain 16:S131-S138 (2000). Examples of causes of nociceptive pain include, but are not limited to, chemical or thermal burns, cuts and contusions of the skin, osteoarthritis, rheumatoid arthritis, tendonitis, and myofascial pain. In certain embodiments, nociceptive pain is stimulated by inflammation.

### III. Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to genetically modified cells of the disclosure or a nucleic acid sequence comprising a synthetic circuit of the disclosure, and delivery systems. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or from a randomized group of subjects. A reference value can be used in place of a control or reference sample, which was previously obtained from a healthy subject or a group of healthy subject. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer implemented method or methods for performing such computer-implemented method or methods, and can also be embodied in the form of a tangible or non-transitory computer readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The terms “patient,” “subject,” “individual,” and the like are used interchangeably herein, and refer to any animal or cells thereof whether in vitro or in situ, amenable to the methods described herein. In certain non-limiting embodiments, the patient, subject or individual is a human.

As used herein, the term “subject” refers to a mammal, preferably a human. The mammals include, but are not limited to, humans, primates, livestock, rodents, and companion animals. A subject may be waiting for medical care or treatment, may be under medical care or treatment, or may have received medical care or treatment.

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host (target) cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

As used herein the term “gene” refers to a polynucleotide (e.g., a DNA segment) that encodes a polypeptide and includes regions preceding and following the coding regions as well as intervening sequences (introns) between individual coding segments (exons). The regions preceding and following the coding regions may comprise regulatory nucleotide sequences such that they are operably linked to the coding regions. “Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 50-99% identity to the reference sequence described herein and screen such for desired phenotypes according to methods routine in the art.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

“Donor DNA”, “donor template” and “repair template” as used interchangeably herein refers to a double-stranded DNA fragment or molecule that includes at least a portion of the gene of interest. The donor DNA may encode a full-functional protein or a partially-functional protein.

“Endogenous gene” as used herein refers to a gene that originates from within an organism, tissue, or cell. An endogenous gene is native to a cell, which is in its normal genomic and chromatin context, and which is not heterologous to the cell. Such cellular genes include, e.g., animal genes, plant genes, bacterial genes, protozoal genes, fungal genes, mitochondrial genes, and chloroplastic genes.

“Functional” and “full-functional” as used herein describes protein that has biological activity. A “functional gene” refers to a gene transcribed to mRNA, which is translated to a functional protein.

“Genetic construct” as used herein refers to the DNA or RNA molecules that comprise a nucleotide sequence that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operably linked to a coding sequence that encodes a protein such that when present in the cell of the individual, the coding sequence will be expressed.

“Genome editing” as used herein refers to changing the endogenous DNA of a cell. Genome editing may include the addition of nucleic acids, deletion of nucleic acids, or restoring a mutant gene. Genome editing may include knocking out a gene, such as a mutant gene or a normal gene, or knocking-in a heterologous gene or protein encoding region thereof.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed or not expressed at all.

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the site specific nuclease, such as with a CRISPR/Cas9-based systems, then the cellular machinery may repair the break by homologous recombination, which is enhanced several orders of magnitude in the presence of DNA cleavage. When the homologous DNA piece is absent, non-homologous end joining may take place instead.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible.

“Nuclease mediated NHEJ” as used herein refers to NHEJ that is initiated after a nuclease, such as a Cas9, cuts double stranded DNA.

“Site-specific nuclease” as used herein refers to an enzyme capable of specifically recognizing and cleaving DNA sequences. The site-specific nuclease may be engineered. Examples of engineered site-specific nucleases include zinc finger nucleases (ZFNs), TAL effector nucleases (TALENs), and CRISPR/Cas-based systems.

“Target region” as used herein refers to the region of the target gene to which the site-specific nuclease is designed to bind and cleave.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA: DNA sequence can be determined using the following formula: Tm=81.5° C.+16.6(log 10[Na+])+0.41 (fraction G/C content)−0.63(% formamide)−(600/1). Furthermore, the Tm of a DNA:DNA hybrid is decreased by 1-1.5ºC for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

“Cell therapy”, “cellular therapy”, or “cytotherapy” as used herein refers to a therapy in which cellular material is administered into a patient or one or more cells in a subject are genetically modified in accordance with the present disclosure. The cellular material may be intact, living cells and provide a therapeutic response to a condition, disease, or disorder in the subject by reducing or preventing one or more symptoms associated with the condition, disease, or disorder; or reduces or prevents progression of the condition, disease, or disorder.

“Chronic disease” as used refers to a long-lasting condition that can be controlled but not cured.

As various changes could be made in the above-described materials and methods without departing from the scope of the invention, it is intended that all matter contained in the above description and in the examples given below, shall be interpreted as illustrative and not in a limiting sense.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: A Synthetic Mechanogenetic Gene Circuit for Autonomous Drug Delivery in Engineered Tissues

Mechanobiologic signals regulate cellular responses under physiologic and pathologic conditions. Using synthetic biology and tissue engineering, the present example provides a mechanically responsive bioartificial tissue that responds to mechanical loading to produce a preprogrammed therapeutic biologic drug. By deconstructing the signaling networks induced by activation of the mechanically sensitive ion channel transient receptor potential vanilloid 4 (TRPV4), synthetic TRPV4-responsive genetic circuits were created in chondrocytes. These cells were engineered into living tissues that respond to mechanical loading by producing the anti-inflammatory biologic drug interleukin-1 receptor antagonist. Chondrocyte TRPV4 is activated by osmotic loading and not by direct cellular deformation, suggesting that tissue loading is transduced into an osmotic signal that activates TRPV4. Either osmotic or mechanical loading of tissues transduced with TRPV4-responsive circuits protected constructs from inflammatory degradation by interleukin-1a. This synthetic mechanobiology approach was used to develop a mechanogenetic system to enable long-term, autonomously regulated drug delivery driven by physiologically relevant loading.

Smart biomaterials or bioartificial tissues that autonomously respond to biologic cues and drive a therapeutic or restorative response are promising technologies for treating both chronic and acute diseases. Mechanotherapeutics, in particular, are a rapidly growing class of smart biomaterials that use mechanical signals or mechanical changes within diseased tissues to elicit a therapeutic response and ameliorate the defective cellular mechanical environment. Current mechanotherapeutic technologies rely on exogenous protein drug delivery or ultrasound stimulation or on synthetic polymer implants that offer a finite life span for drug delivery. Creating systems with cellular-scale resolution of mechanical forces that offer long-term, feedback-controlled synthesis of biologic drugs could provide a completely new approach for therapeutic delivery.

In contrast to synthetic polymers, biological tissues grow, adapt, and respond to mechanobiologic signals through the use of specialized molecular components, such as mechanically-sensitive ion channels and receptors that transduce specific stimuli from the physical environment. In particular, mechanosensitive ion channels are sensitive to both context and deformation mode, making them uniquely suited as mechanotherapeutic sensors. The transient receptor potential (TRP) family is a class of selective ion channels including some mechanically sensitive members, such as TRPA1, TRP vanilloid 1 (TRPV1), and TRPV4. TRPV4 is activated by osmotic stress and plays an important role in the mechanosensitive of various tissues such as articular cartilage, uterus, and skin. In cartilage, TRPV4 has been shown to regulate the anabolic biosynthesis of chondrocytes in response to physiologic mechanical strain.

Osteoarthritis is a chronic joint disease for which there are no available disease-modifying drugs, ultimately leading to a total joint replacement once the diseased and degraded cartilage and surrounding joint tissues incapacitate a patient from pain and a loss of joint function. Cartilage tissue engineering is a promising strategy to resurface damaged and diseased articular cartilage with an engineered cartilage tissue construct as a means to reduce the need for, or prolong the time before, a total joint replacement. An ongoing challenge in the field, however, is developing engineered cartilage constructs that withstand both the high mechanical loads present within the articular joint and the chronic inflammation present within an osteoarthritic joint. For example, the knee cartilage of healthy individuals can experience compressive strains of ˜5 to 10% during moderate exercise, while individuals with a history of joint injury or high body mass index, populations at risk for developing osteoarthritis, can experience higher magnitudes of cartilage compression under similar loading. In this regard, a bioartificial tissue that can synthesize biologic drugs in response to inflammation or mechanical loading, either independently or concurrently, could greatly enhance the therapeutic potential of an engineered tissue replacement.

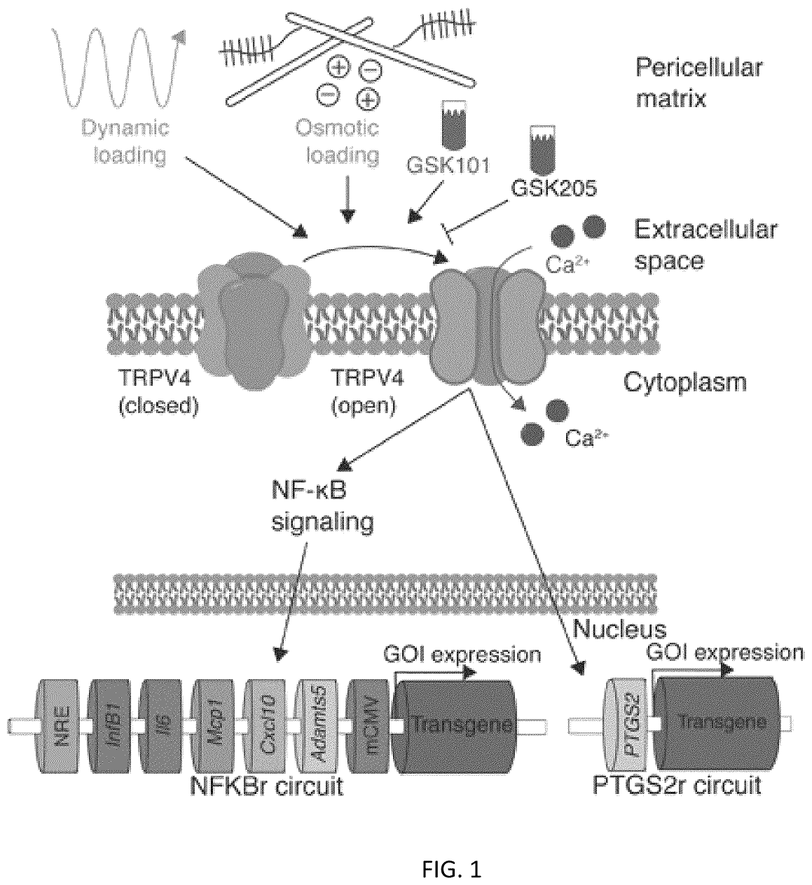

The present example provides a mechanically responsive bioartificial tissue construct for therapeutic drug delivery by using the signaling pathways downstream of the mechano/osmosensitive ion channel TRPV4 to drive synthetic mechanogenetic gene circuits (FIG. 1). To do this, mechanical loading of tissue-engineered cartilage was first established to activate TRPV4 through fluctuations in the local osmotic environment and not direct mechanical deformation of chondrocytes. Next, the gene regulatory networks (GRNs) were deconstructed and signaling pathways evoked by TRPV4 activation and revealed the transient activation of several mechanosensitive transcription factors. Synthetic gene circuits were engineered to respond to mechanical TRPV4 activation for driving transgene production of an anti-inflammatory molecule, interleukin-1 receptor antagonist (IL-1Ra). While IL-1Ra (drug name anakinra) is approved as a therapy for rheumatoid arthritis and has successfully attenuated osteoarthritis progression in preclinical models, clinical trials of IL-1Ra therapy in patients with established osteoarthritis have not shown efficacy, suggesting that controlled long-term delivery may be necessary for disease modification. The present example shows that mechanical or osmotic loading of implantable engineered cartilage tissue constructs induces an autonomous mechanogenetic response and protects tissue constructs from inflammatory insult, suggesting a modality for long-term in vivo drug delivery.

### Results

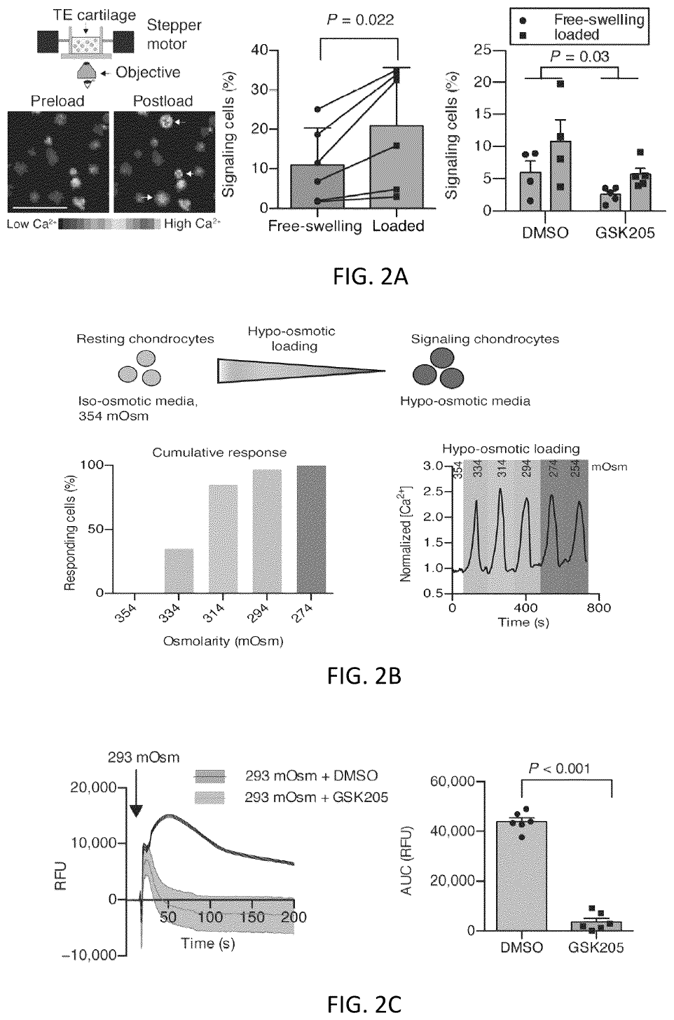

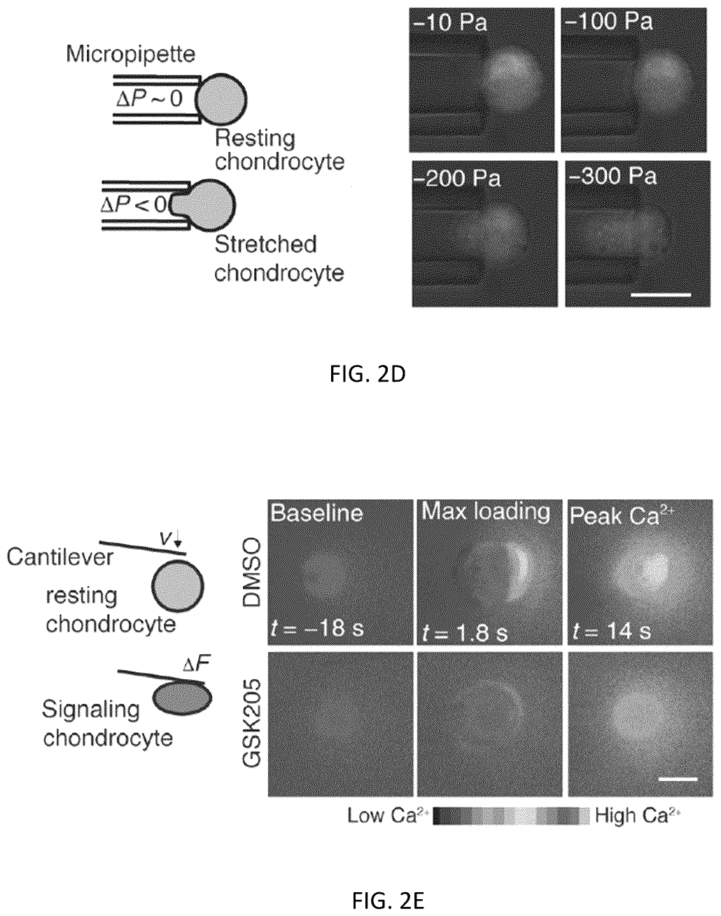

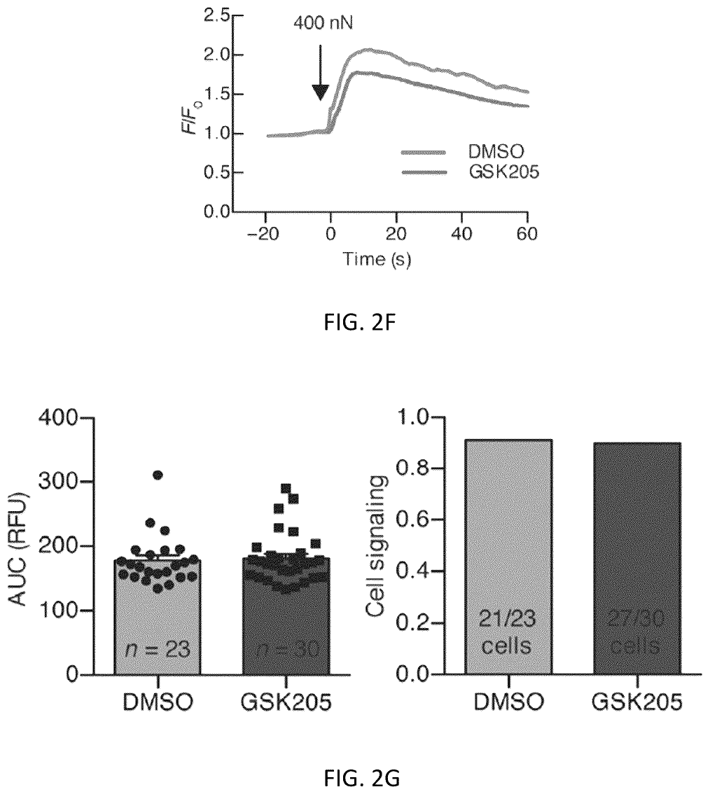

The mechano-osmotic response of chondrocytes to loading is regulated by TRPV4: To deconstruct the mechanotransduction pathways through which chondrocytes perceive mechanical loading, freshly isolated primary porcine chondrocytes were encapsulated within an agarose hydrogel scaffold to create engineered cartilage tissue constructs. Constructs were cultured for 3 weeks to allow extracellular matrix deposition before applying compressive loading and simultaneously imaging the intracellular calcium levels of the chondrocytes. This engineered cartilage system allows mechanical signals to be transduced to chondrocytes through de novo synthesized extracellular matrix in a manner similar to in vivo mechanotransduction. Chondrocytes express an array of mechanically sensitive ion channels and receptors, including TRPV4, PIEZO1 and PIEZO2, integrins, and primary cilia, so the mechanism by which physiologic magnitudes of dynamic mechanical loading are transduced by chondrocytes were investigated. Dynamic mechanical loading of engineered cartilage constructs immediately provoked a 108% increase of intracellular calcium response compared with unloaded tissues (P=0.022; FIG. 2A). Inhibiting TRPV4 activity using the TRPV4 antagonist GSK205 suppressed calcium signaling (P=0.03, GSK205 supplementation reduced signaling by ˜47%), implicating TRPV4 as a critical component in transducing compressive loading in chondrocytes.

While the molecular structure of TRPV4 has recently been reported, the mechanisms underlying TRPV4 activation are complex and may involve direct mechanical activation or osmotic activation secondary to mechanical loading of the charged and hydrated extracellular matrix. To determine the physical mechanisms responsible for TRPV4 activation secondary to tissue compression, freshly isolated chondrocytes were subjected to osmotic loading, direct membrane stretch, and direct single-cell mechanical compression (FIG. 2B, FIG. 2D, and FIG. 2E). To better understand the biophysical mechanisms underlying TRPV4 activation, the mechanical state of the chondrocyte membrane in each of these cases were determined using finite element modeling (FEBio; febio.org). Chondrocytes rapidly responded to physiologically relevant changes in medium osmolarity through intracellular calcium signals (FIG. 2B). Calcium signaling was highly sensitive to modest changes in osmolarity, with an osmolarity shift of −20 mOsm inducing intracellular signaling in 35% of chondrocytes and −80 mOsm inducing activation of 100% of chondrocytes. Inhibition of TRPV4 with GSK205 suppressed calcium signaling caused by hypo-osmotic loading (FIG. 2C) or by the TRPV4 agonist GSK1016790A (GSK101). Chondrocyte volumetric analysis and finite element modeling of hypo-osmotic stress showed that a change of −60 mOsm, sufficient to induce signaling in nearly all chondrocytes, increased cellular volume by 13% and induced an apparent first-principle strain of 0.04 homogeneously throughout the membrane. To investigate the role of direct membrane stretch on TRPV4 activation in the absence of osmotic fluctuations, micropipette aspiration of individual chondrocytes was used to apply controlled deformation of the cell membrane (FIG. 2D). Unexpectedly, micropipette aspiration did not provoke any calcium signaling response in chondrocytes, with only 1 of 38 tested cells responding with increased intracellular calcium, indicating that membrane deformation per se was not the primary signal responsible for mechanical activation. Finite element simulations of the micropipette experiment showed the presence of heterogenous apparent membrane strains in aspirated chondrocytes, with a first-principle strain reaching 0.31 around the micropipette mouth and ˜0.04 within the micropipette under an applied pressure of 100 Pa. To test whether calcium signaling in response to direct mechanical compression of chondrocytes is mediated by TRPV4, isolated chondrocytes were loaded with an atomic force microscope (AFM) to 400 nN. Only at high, pathologic levels of mechanical compression did direct chondrocyte compression provoke intracellular calcium signaling, but this response was not inhibited by GSK205 (FIG. 2E to FIG. 2G). Finite element modeling predicted high membrane strains with an ultimate apparent first-principle membrane strain of 2.27 around the cell at the peak cell deformations necessary to elicit intracellular calcium signaling. Together, these findings suggest that activation of TRPV4 in chondrocytes in situ may not result directly from cellular strain but rather from osmotic fluctuations induced from the deformation of a chondrocyte's osmotically active environment. Because of the complex relationship between deformational compressive loading of engineered cartilage and osmotic loading, mechanical loading will hereafter refer to deformational compressive loading (concomitant with the secondary osmotic effects) and direct changes in the medium osmolarity as osmotic loading.

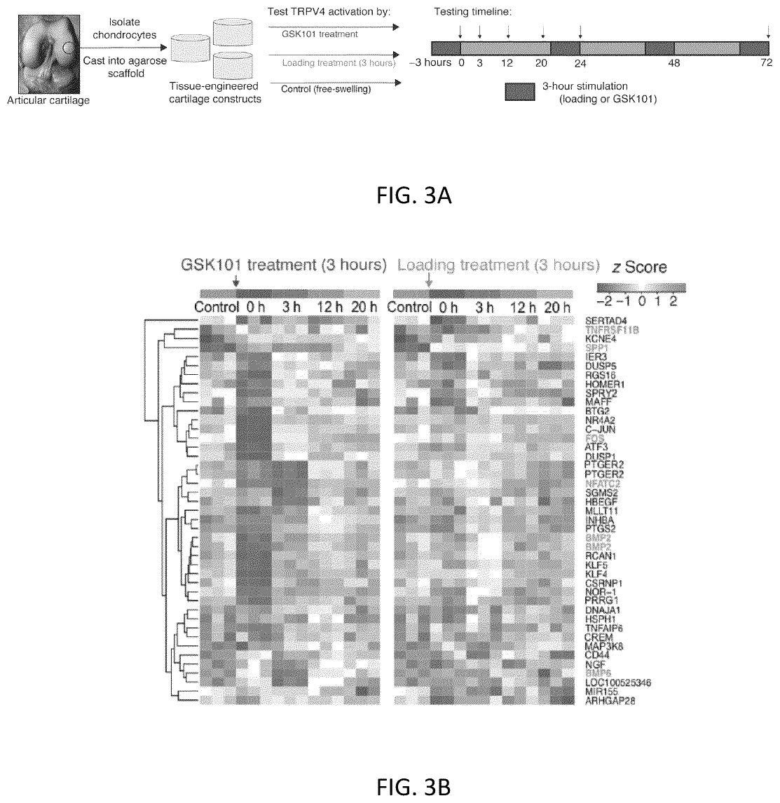

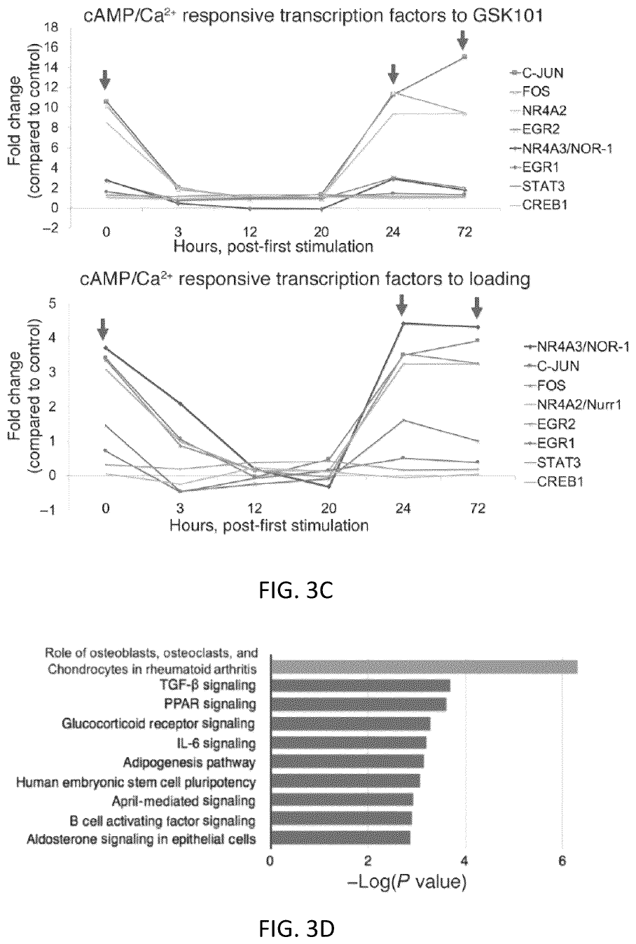

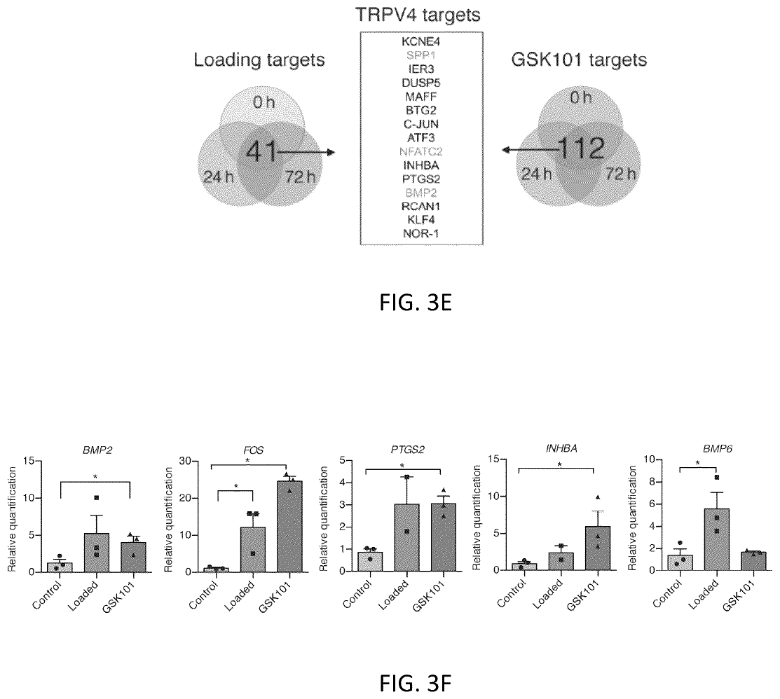

TRPV4 activation in chondrocytes induces transient anabolic and inflammatory signaling networks: Next, it was sought to understand the time course of specific downstream signaling pathways and GRNs regulated by mechanical activation of TRPV4 using microarray analysis. Because TRPV4 is a multimodal channel, downstream signaling is likely dependent on the activation mode as well as cell and tissue type. For these studies, primary porcine chondrocytes were cast in agarose to create engineered cartilage constructs and subjected to either compressive mechanical loading [10% sinusoidal peak-to-peak strain at 1 Hz for 3 hours as described previously] or pharmacologic stimulation with the TRPV4 agonist GSK101 (1 nM for 3 hours), or left unloaded (free-swelling controls), and measured the transcriptional activity at 0, 3, 12, and 20 hours following an initial round of stimulation and then at 24 and 72 hours after additional daily bouts of mechanical loading or GSK101 (n=3 per condition per time point; FIG. 3A). In the first 20 hours following initial stimulation, there were 43 transcripts up-regulated under both mechanical loading and pharmacologic stimulation (FIG. 3B) compared to unloaded controls. Up-regulation of these targets was immediate and subsided quickly, with transcript levels returning to baseline 12 to 20 hours after activation. In particular, the adenosine 3′,5′-monophosphate (CAMP)/Ca²⁺-responsive transcription factors C-JUN, FOS, NR4A2, and EGR2 (FIG. 3C) were all up-regulated in response to TRPV4-mediated calcium signaling.

The Ingenuity Pathway Analysis was then used to identify candidate targets for a synthetic gene circuit that would be responsive to TRPV4 activation. The most enhanced pathway was “the role of osteoblasts, osteoclasts, and chondrocytes in rheumatoid arthritis,” with members including transcription factors (FOS and NFATC2), extracellular matrix synthesis gene (SPP1), growth factors (BMP2 and BMP6), and TNFSF11, the decoy ligand for RANKL, all of which were up-regulated by TRPV4 activation (FIG. 3D). The inflammatory pathways “IL-6 signaling” and “B cell activating factor signaling” were also significantly up-regulated by TRPV4 activation, as were the established chondrocyte anabolic pathways “TGF-β signaling” and “glucocorticoid signaling.” Because of the rapid response times of nearly all of the differentially expressed genes (DEGs), which genes responded to repeated bouts of TRPV4 stimulation were further analyzed at all three time points (0, 24, and 72 hours). There were 41 transcripts repeatedly responsive to mechanical loading and 112 transcripts repeatedly responsive to GSK101 supplementation. Of these targets, 15 genes were commonly responsive to both mechanical loading and pharmacologic activation of TRPV4 (FIG. 3E) and include both pathways associated with inflammation (PTGS2, SPP1, and ATF3) and cartilage development and homeostasis (BMP2, DUSP5, NFATC2, and INHBA). The most robustly regulated and cartilage-relevant genes were confirmed by quantitative polymerase chain reaction (qPCR) (FIG. 3F). Together, these data suggest that the TRPV4 mechanotransduction pathway involves activation of a rapidly resolving inflammatory pathway as part of the broad anabolic response to physiologic mechanical loading.

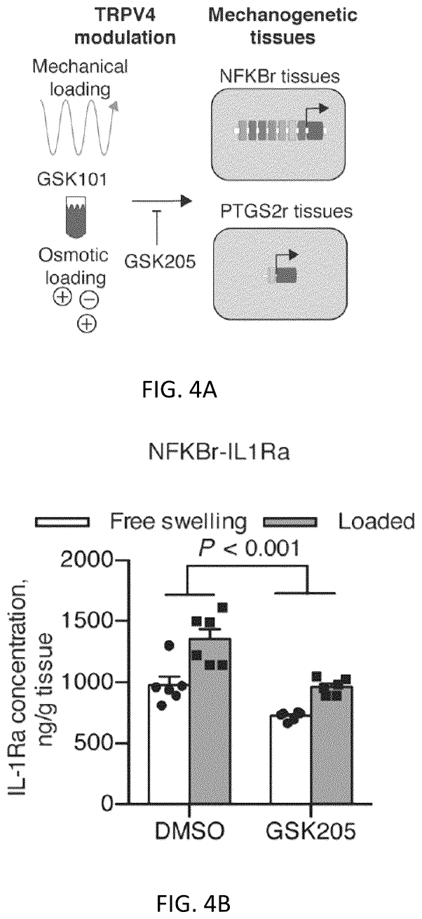

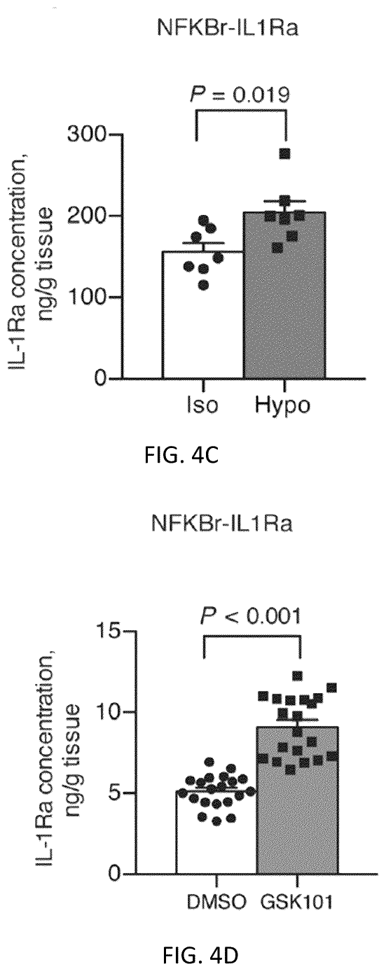

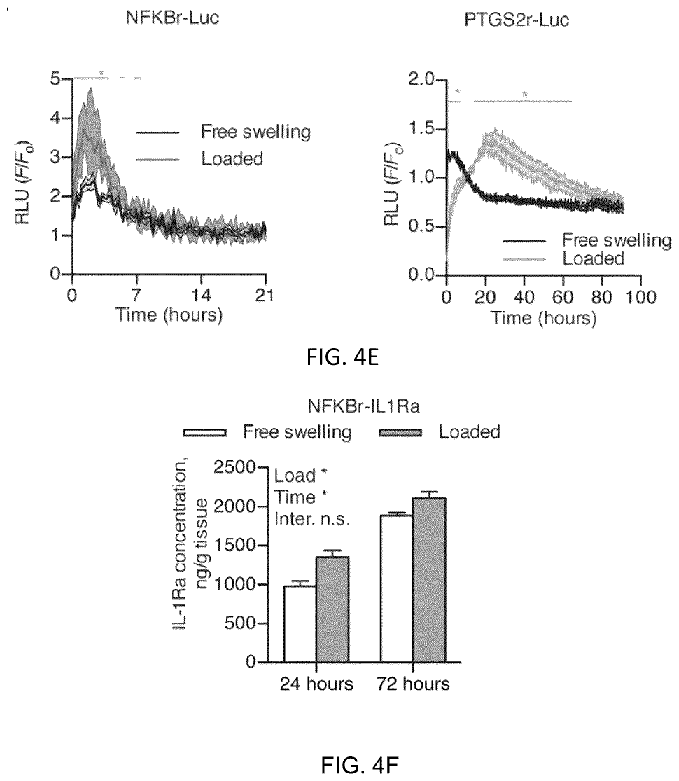

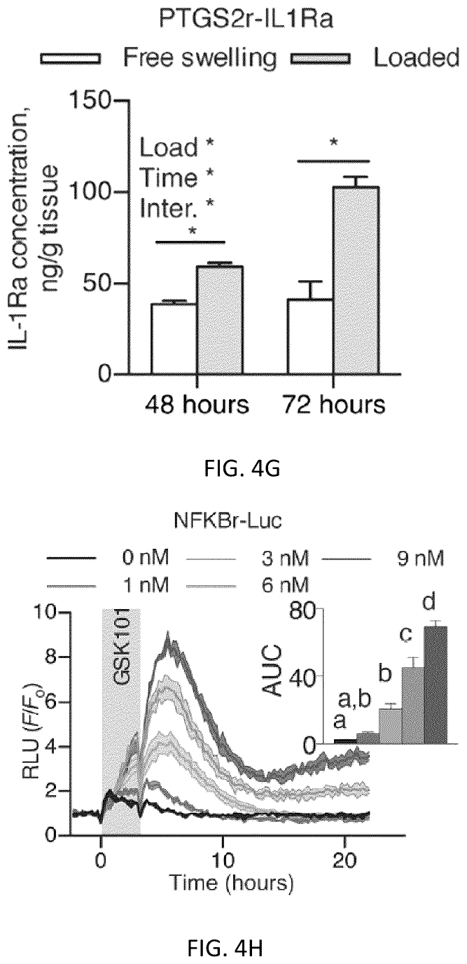

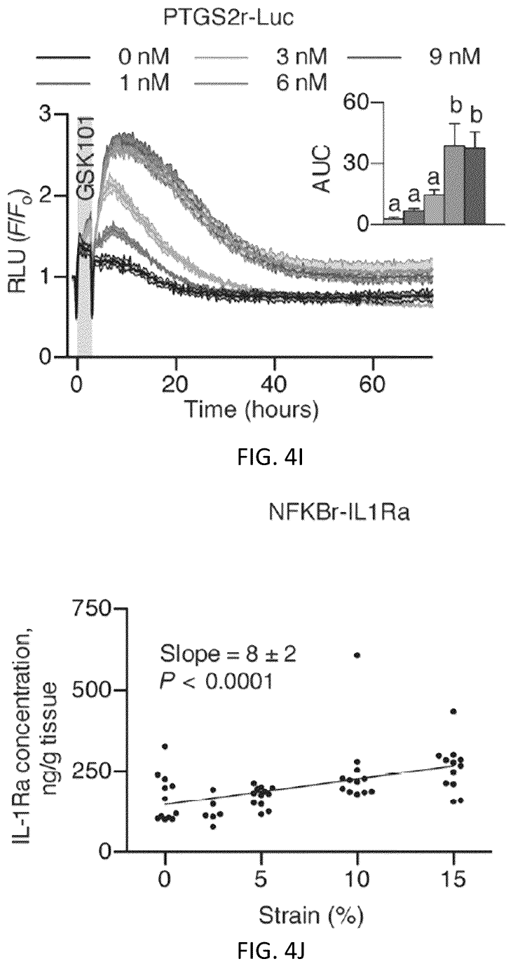

Synthetic mechanogenetic circuits respond to TRPV4 activation to drive transgene expression: TRPV4 activation in response to mechanical loading up-regulates a diverse group of targeted genes. On the basis of the TRPV4-activated signaling pathway, activation of the nuclear factor κ light chain enhancer of activated B cells (NF-κB) pathway was identified and up-regulation of the prostaglandin-endoperoxide synthase 2 (PTGS2) gene as two distinct avenues to construct TRPV4-responsive synthetic mechanogenetic gene circuits. Targeting the NF-κB signaling pathway and regulation of the PTGS2 promoter (FIG. 1), two lentiviral systems were developed that would either (i) respond to NF-κB activity by linking five synthetic NF-κB binding motifs and a NF-κB-negative regulatory element with the cytomegalovirus (CMV) enhancer to drive transgene expression of either the therapeutic anti-inflammatory biologic IL-1Ra or a luciferase reporter (henceforth referred to as NFKBr-IL1Ra and NFKBr-Luc, respectively) or (ii) respond to PTGS2 regulation by using a synthetic human PTGS2 promoter to drive either IL-1Ra or luciferase expression (henceforth referred to as PTGS2r-IL1Ra and PTGS2r-Luc, respectively). mechanogenetically sensitive engineered cartilage tissue constructs were then created by lentivirally transducing primary porcine chondrocytes with a mechanogenetic circuit and seeding these cells into an agarose hydrogel to produce synthetically programmed cartilage constructs (FIG. 4A).

To test whether mechanogenetic tissues actively respond to mechanical stimulation for transgene production, compressive mechanical loading was first applied to NFKBr-IL1Ra-transduced mechanogenetic cartilage constructs, which showed a 38% increase in IL-1Ra production (P=0.006) with mechanical loading (FIG. 4B) compared to free-swelling controls. To establish whether this response was dependent on TRPV4, TRPV4 was antagonized with GSK205 and observed a significant attenuation of NFKBr-IL1Ra circuit activity (FIG. 4B), demonstrating that the mechanogenetic circuit responded to both mechanical loading (P<0.001) and TRPV4 antagonism (P<0.001). GSK205 also reduced circuit activation in unloaded tissues, suggesting that TRPV4 activation in chondrocytes may not be entirely dependent on mechanical loading. To further assess the specificity of TRPV4 regulation in mechanogenetic cartilage constructs, direct hypo-osmotic loading was applied and pharmacologic GSK101 stimulation to NFKBr-IL1Ra tissues. A step change to hypo-osmotic medium (−200 mOsm change) activated the NFKBr-IL1Ra mechanogenetic circuit (P=0.019; FIG. 4C) compared to an iso-osmotic medium change (0 mOsm change). In addition, daily pharmacologic activation of TRPV4 with 1 nM GSK101 for 3 hours/day over the course of 5 days activated the NFKBr-IL1Ra circuit compared to dimethyl sulfoxide (DMSO) controls (P<0.001; FIG. 4D). Engineered cartilage constructs responded repeatedly and reproducibly to the daily GSK101 stimulation, demonstrating the role of TRPV4 in activating the mechanogenetic NFKBr circuits and confirming that a cell-based mechanotherapy can offer prolonged and unabating biologic drug delivery. Testing of conditioned medium from constructs seeded with either nontransduced chondrocytes or chondrocytes transduced with a green fluorescent protein (GFP) expression cassette found that only cells transduced with a mechanogenetic IL-1Ra-producing circuits were capable of synthesizing detectable levels of IL-1Ra.

To determine the sensitivity and temporal response kinetics of the engineered tissue system, factors critical for the effectiveness of any drug delivery system, bioluminescence imaging of NFKBr-Luc or PTGS2r-Luc constructs was used to determine the dynamic response of mechanogenetic cartilage constructs to TRPV4 activation by mechanical loading or GSK101 pharmacologic stimulation. In response to 10% compressive mechanical loading (FIG. 4E), NFKBr-Luc tissue constructs rapidly peaked (1.8±0.2 hours to peak) and decayed (T50% decay time=3.4±0.4 hours) with loaded samples returning to baseline 4 hours after loading. PTGS2r-Luc tissue constructs were slower to activate (21.7±2.7 hours to peak) and remained activated for a longer duration (T50% decay time=22.3±1.7 hours). This differential in time delivery kinetics may provide strategies by which mechanical loading inputs can drive both short- and long-term drug production by judicious mechanogenetic circuit selection in a single therapeutic tissue construct. To test whether IL-1Ra production followed similar differential production rates, NFKBr-IL1Ra and PTGS2r-IL1Ra tissue constructs were mechanically loaded and measured protein levels of IL-1Ra released in the medium. One round of mechanical loading activated NFKBr-IL1Ra tissues by 24 hours, as measured by an increase in IL-1Ra produced by loaded tissue constructs compared to unloaded constructs (an increase in IL-1Ra production of 372±265 ng/g, means±1 SD). This differential in IL-1Ra concentration between loaded and free-swelling constructs remained unchanged by 72 hours (loaded tissues produced 220±223 ng/g more than unloaded constructs at this time point), indicating that loaded NFKBr tissues were not continually activated and resumed baseline activity levels after 24 hours (FIG. 4F). Conversely, in a preliminary experiment, a single round of mechanical loading did not differentially activate PTGS2r-IL1Ra in the 24 hours after loading. Informed by the bioluminescent imaging, conditioned medium of PTGS2r-IL1Ra tissues 48 and 72 hours after a single round of mechanical loading was measured, however, it was found that tissues produced more IL-1Ra after 48 hours compared to free-swelling tissues (difference in means of 21 ng/g) and that the IL-1Ra difference between loaded and free-swelling tissues continued to increase by 72 hours (difference in means of 61 ng/g). This increased effect size confirmed that mechanically loaded PTGS2r tissue constructs remained activated up to 72 hours after loading and demonstrated a longer-acting mechanogenetic response in the PTGS2r system (FIG. 4G). On the basis of the bioluminescent imaging results, IL-1Ra levels past 72 hours were not measured. These results show that complex drug delivery strategies can be programmed into a single mechanogenetic tissue constructs to produce multiple modes and time scales of therapeutic or regenerative biologic drug delivery.

For insight into the dose response relationship and sensitivity of the mechanogenetic gene circuits, NFKBr-Luc and PTGS2r-Luc bioluminescence was imaged in response to different doses of GSK101. Temporal imaging revealed that GSK101 stimulation produced similar rise and decay kinetics as TRPV4 activation from mechanical loading (FIG. 4E) in NFKBr-Luc (FIG. 4H) and PTGS2r-Luc (FIG. 4I) cartilage tissue constructs compared to unstimulated controls. Both mechanogenetic tissue constructs displayed a clear dose-dependent activation from GSK101 stimulation as demonstrated by the AUC (area under the curve). NFKBr-Luc tissue constructs were responsive from 1 to 9 nM GSK101, whereas PTGS2r-Luc tissue constructs plateaued in response at 6 nM GSK101. On the basis of this pharmacologic sensitivity, it was hypothesized that mechanogenetic constructs would be increasingly activated by higher mechanical loading strains through increased osmotic stimulation (FIG. 2B). As NFKBr-Luc tissues demonstrated the most pronounced GSK101 dose-dependent response, dynamic compressive strain amplitudes from 0 to 15% was applied to NFKBr-IL1Ra tissue constructs to span the range of physiologic strains expected for articular cartilage in vivo. Elevated production of IL-1Ra was measured with increasing compressive strains (slope=8±2 ng/g IL-1Ra per % strain, P<0.001; FIG. 4J), demonstrating that drug production is directly responsive to the magnitude of mechanical loading in our mechanogenetic engineered tissues.

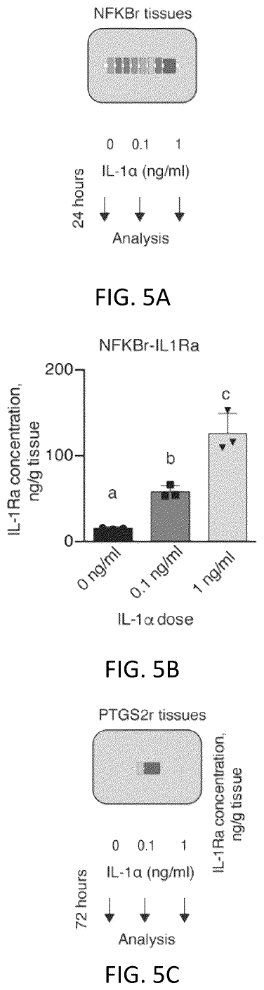

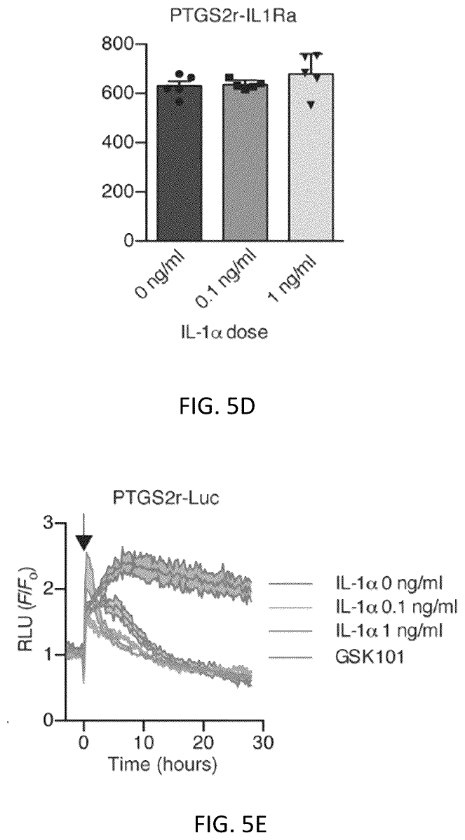

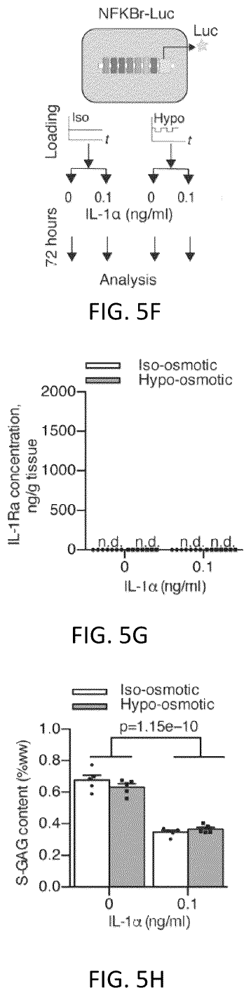

Mechanogenetic engineered cartilage activation protects cartilage tissues from IL-1α-induced inflammation-driven degradation: The long-term success of engineered cartilage implants depends on the ability of implants to withstand the extreme loading and inflammatory stresses within an injured or osteoarthritic joint. It was hypothesized that TRPV4 activation would activate mechanogenetic tissue constructs to produce therapeutic levels of IL-1Ra and protect engineered cartilage constructs and the surrounding joint from destructive inflammatory cytokines. As our mechanogenetic circuits rely on signaling pathways that overlap with the cellular inflammatory response, the dose response of IL-1Ra production was examined in response to the inflammatory cytokine IL-1a (FIG. 5A and FIG. 5C). NFKBr-IL1Ra mechanogenetic constructs responded to exogenous IL-1α supplementation following a dose-dependent characteristic, consistent with findings of IL-1-induced NF-κB signaling in chondrocytes (FIG. 5B). This dose-dependent response to IL-1α was present up to 10 ng/ml and offered prolonged and robust production of IL-1Ra, promoting the notion that cell-based tissues may offer a more sustained ability to produce therapeutic biomolecules relative to traditional acellular approaches. Mechanical loading further enhanced IL-1Ra production in the presence of IL-1α, demonstrating that, even in the presence of high levels of inflammation, mechanical loading further potentiates NF-κB signaling. Mechanogenetic PTGS2r-IL1Ra and PTGS2r-Luc constructs demonstrated no sensitivity to IL-1α (FIG. 5D and FIG. 5E). In contrast to NFKBr tissue constructs, the selective sensitivity to TRPV4 activation and not to IL-1α in PTGS2r tissue constructs suggests that this system is distinctively sensitive to mechano- or osmo-activation of TRPV4. This distinction was further demonstrated in tissues exposed to both IL-1α inflammation and osmotic loading, wherein only loading played a significant influence (P=0.0002 for loading and P=0.14 for inflammation).

To test whether TRPV4 activation-induced IL-1Ra production would protect engineered mechanogenetic tissue constructs against IL-1α, mature, 21-day-old NFKBr tissue constructs were cocultured with articular cartilage explants in the presence of IL-1α inflammation and daily hypo-osmotic loading (incubation in 180 mOsm medium for 3 hours/day; standard iso-osmotic medium was maintained at 380 mOsm) to model the inflammation and osmotic conditions present in the cartilage of arthritic joints exposed to daily loading (FIG. 5F and FIG. 5J). Using NFKBr-Luc tissue constructs, which lack an anti-inflammatory response to TRPV4 or IL-1α activation (FIG. 5G), to simulate how conventional engineered cartilage constructs would respond in an arthritic joint, constructs lost 45.6% of their sulfated glycosaminoglycan (S-GAG) content after 72 hours of IL-1α treatment (FIG. 5H). S-GAGs are essential structural molecules that impart mechanical integrity and strength in both engineered and native cartilage. Osmotic loading did not modulate this response, and the substantial S-GAG loss was observable histologically through diminished safranin-O staining throughout the tissue (FIG. 5I). In the anti-inflammatory NFKBr-IL1Ra tissue constructs, osmotic loading in the presence of inflammatory IL-1α increased IL-1Ra production by 93% over iso-osmotic control tissues also cultured with IL-1α (FIG. 5K). After 72 hours of treatment, IL-1α induced a 30.8% loss of S-GAG in NFKBr-IL1Ra-engineered cartilage constructs under iso-osmotic conditions, while NFKBr-IL1Ra-engineered cartilage that was incubated with IL-1α and osmotically loaded did not significantly lose their S-GAG content (FIG. 5L). Histologically, hypo-osmotic loading NFKBr-IL1Ra-engineered cartilage constructs maintained rich safranin-O staining of S-GAG throughout the constructs, even in the presence of IL-1α (FIG. 5M). Explant proteoglycan levels were similar across all IL-1α treatment groups. Together, these data demonstrate engineered mechanogenetic cartilage constructs that can produce anti-inflammatory IL-1Ra in response to osmotic loading at levels sufficient for engineered tissue protection in a proinflammatory environment mimicking the conditions present in an osteoarthritic joint.

### Discussion

By combining synthetic biology and tissue engineering, a novel class of bioartificial material was developed that is mechanogenetically sensitive. This system functions by redirecting endogenous mechanically sensitive ion channels to drive synthetic genetic circuits for converting mechanical inputs into programmed expression of a therapeutic transgene. By engineering these cells into a functional tissue construct, this system provides the potential to repair or resurface damaged cartilage while providing site-specific, mechanically induced anti-cytokine therapy against inflammation. This approach is based on redirection of the downstream response to activation of the TRPV4 ion channel—a critical mechanosensor in cartilage—to transduce tissue-scale deformational mechanical loading via mechano-osmotic coupling in the charged extracellular matrix. By deconstructing the GRNs activated by mechanically induced TRPV4, chondrocyte-endogenous signaling machinery were coopted to drive synthetic circuits for the production of therapeutic biologic drugs, using the anti-inflammatory drug IL-1Ra as a model system for proof of concept. These results also demonstrate the use of distinct signaling networks for defining the specificity, timing, and dose response for the expression of therapeutic biologic drugs. The present example shows that a single round of mechanical loading can induce short-term and long-term responses based on the particular response of the synthetic circuit. While a treatment for cartilage repair and osteoarthritis were targeted, pathologic mechanical loading and mechano-signaling play a role in a broad range of acute and chronic diseases (12), suggesting a wide range of potential therapeutic applications for mechanogenetically regulated cells or tissues requiring autonomous cellular control systems.

While mechanogenetic cartilage tissues were developed based on TRPV4 activation here, the use of other native, mechanically sensitive ion channels and receptors provides an attractive source of mechanosensors that can be elicited to provoke synthetic outputs. Expanding mechanogenetic approaches to additional mechanosensors with applications to other tissues would increase the range of physical stimuli that synthetic circuits can respond to but requires an in-depth understanding of both the mechanical contexts necessary for mechanosensor activation and the resulting downstream signaling pathways. The fact that primary chondrocytes have an array of different mechanically sensitive ion channels and receptors highlights the potential opportunities to layer mechanosensor-specific circuits and produce systems responsive to different mechanical inputs that drive specific synthetic outputs. The analysis here demonstrates that physiologic (˜10%, 1 Hz) mechanical loading of engineered cartilage is converted to a mechano-osmotic signal that activates TRPV4, evidenced by the GSK205 inhibition of chondrocyte calcium signaling in response to mechanical loading or osmotic loading of engineered cartilage but not to direct cellular deformation or membrane deformation. To this end, the mechanoresponsiveness of TRPV4 signaling was examined and two genetic circuits developed that respond to TRPV4 activation. Note that while endogenous PTGS2 regulation subsided within 24 hours after mechanical loading, our PTGS2r circuit remained activated up to 72 hours after loading, highlighting the role that endogenous mechanisms of gene regulation may play, which are likely absent in our synthetic circuits. In this regard, the use of alternate chondrocyte mechanosensors remains an attractive area of research. For instance, the lack of a TRPV4-dependent response to high-strain compression via AFM loading is consistent with our earlier finding that high, potentially injurious, cellular strains are transduced via the PIEZO family of ion channels. The multimodal nature of TRPV4, and the TRP family more generally, suggests that additional genetic circuits may be developed for alternate activation modes by characterizing the downstream signaling networks that respond to temperature or biochemical activation of the channel. Moreover, engineering novel mechanosensors may open the opportunity for developing custom mechanical activation modes and orthogonal downstream signaling in future mechanogenetic systems.

These findings offer a detailed perspective of the complex cellular events initiated by TRPV4 activation in chondrocytes. TRPV4 has been thought to play a largely anabolic role in chondrocytes through enhanced synthesis of matrix molecules, S-GAG and collagen, and up-regulation of transforming growth factor-β (TGF-B3), which is evident in our Gene Ontology (GO)/Kyoto Encyclopedia of Genes and Genomes (KEGG) pathway hits of “TGF-B signaling” and “glucocorticoid receptor signaling.” However, these findings also revealed the presence of an acute, transiently resolving inflammatory response (pathways including “chondrocytes in RA,” “IL-6 signaling,” “April-mediated signaling,” and “B cell activating factor signaling”). These findings are particularly striking, as the physiologic levels of mechanical loading applied here (10% compressive strain) are typically associated with promoting chondrocyte anabolism, although these data are consistent with other reports suggesting a proinflammatory role of TRPV4. Together, the rapidly resolving mechanical load-induced inflammation in this context may be part of a natural cascade by which a quickly decaying inflammatory response is characteristic of the regenerative, anabolic response to mechanical loading. In addition, increasing evidence suggests a potential role for TRPV4 in mediating cellular and tissue inflammation, and studies of chondrocyte-specific TRPV4 knockout mice report decreased severity of age-associated osteoarthritis. A load-induced mechanism of chondrocyte inflammation through TRPV4 may provide a target for osteoarthritis and other age-related diseases. The synthetic gene circuits developed in this study highlight the opportunities to target different responses through downstream pathway selection, namely, using an NFKBr circuit that is sensitive to IL-1α inflammation and a PTGS2r circuit that is IL-1α insensitive. To this end, the pathologic conditions present in osteoarthritis generate a milieu of inflammatory agents and factors that may, in addition to TRPV4 activation, induce NF-κB signaling and PTGS2 regulation. Deep RNA sequencing and promotor engineering may provide a unique strategy for developing distinct, mechanoresponsive tools in an inflammatory, osteoarthritic joint. Additional therapeutic targets can also be readily inhibited or activated as well; previous work investigated using intracellular inhibitors of NF-κB signaling and the soluble tumor necrosis factor receptor-1 as two alternative options for inhibiting inflammation.

Using an engineered, living tissue construct for coordinated drug delivery obviates many of the traditional limitations of “smart” materials, such as long-term integration, rapid dynamic responses, and extended drug delivery without the need for replacement or reimplantation of the drug delivery system. The modular approach of using cells as both the mechanosensors and the effectors within engineered tissues allows regulation and sensitivity at the mechanically sensitive channel or receptor level, the signal network level, and the gene circuit level. While the primary chondrocyte's endogenous TRPV4 to drive was used the exemplary synthetic system of this example, engineering novel mechanically sensitive proteins may open a new frontier for coordinating inputs or driving receptor activation from novel and precise mechanical inputs. Together, this framework for developing mechanically responsive engineered tissues is a novel approach for establishing new autonomous therapeutics and drug delivery systems for mechanotherapeutics.

### Methods

Tissue harvest, cell isolation, agarose gel casting, and culture: Full-thickness porcine articular chondrocytes were enzymatically isolated using collagenase (Sigma-Aldrich) from the femurs of pigs obtained from a local abattoir (˜30 kg; 12 to 16 weeks old) post mortem in accordance with an exemption protocol by the Institutional Animal Care and Use Committee. Filtered cells were mixed 1:1 with 4% molten type VII agarose (Sigma-Aldrich), and the cell-agarose mixture was injected into a gel apparatus and allowed to set at room temperature. Chondrocyte-laden disks were punched out, yielding engineered cartilage at a final concentration of 2% agarose and 15 to 20 million cells/ml. All constructs were given 2 to 3 days to equilibrate, and media were changed three times per week during chondrogenic culture using a base medium that consisted of Dulbecco's modified Eagle's medium (DMEM) High Glucose (Gibco) supplemented with 10% fetal bovine serum (FBS) (Atlas), 0.1 mM nonessential amino acids (Gibco), 15 mM Hepes (Gibco), proline (40 μg/ml) (Sigma-Aldrich), 1×penicillin/streptomycin, fungizone (Gibco), and fresh ascorbyl-2-phosphate (50 μg/ml) (Sigma-Aldrich) and maintained at 37° C. and 5% CO₂.

Chondrocyte mechanical and pharmacologic stimulation: Confocal imaging of mechanical compression—A custom mechanical compression device was used to compress agarose constructs while simultaneously performing intracellular calcium imaging on a confocal microscope (Zeiss LSM 880) using fluo-4 AM and fura-red AM (Thermo Fisher Scientific) based on the manufacturer's protocols. Opposing platens were controlled with stepper motors to apply 60 rounds of compressive loading (10%) after a 2% tare strain. Ratiometric calcium imaging (Calcium Ratio=Intensityfluo-4/Intensityfura-red) was analyzed within each sample with ImageJ for 2.5 min in before and for 2.5 min after the mechanical loading.

High-throughput mechanical compression—A custom mechanical compression device was used for sinusoidal compression of 24 individual tissue constructs simultaneously using a closed-loop displacement feedback system. This system allows compression at 37° C. and 5% CO₂.

Osmotic stimulation—For calcium imaging studies, osmotic loading media were prepared using Hanks' balanced salt solution medium (Gibco). For mechanogenetic tissue culture studies, osmotic loading media were prepared using DMEM with 1% insulin/transferrin/selenous acid premix (ITS+, Corning, #354352). These media were titrated to hypo-osmotic media by adding distilled water and measured with a freezing point osmometer (Osmette 2007; Precision Systems). For osmotic stimulation of mechanogenetic samples, standard culture medium (containing 10% FBS) was replaced with iso-osmotic (380 mOsm) ITS+base medium, containing 1% ITS+premix (Corning), 0.1 mM nonessential amino acids, 15 mM Hepes, and 1×penicillin/streptomycin, 3 days before osmotic loading to acclimate tissues.

Micropipette aspiration—Detailed methods for micropipette aspiration are described previously. Briefly, glass micropipettes were drawn to a diameter of ˜10 μm and coated with Sigmacote (Sigma-Aldrich) to prevent cell binding to the glass micropipette. The micropipette was brought in contact with a cell, and a tare pressure of 10 Pa was applied for a period of 3 min. Increasing step pressures were then applied in increments of 100 Pa for 3 min each until the cell was fully aspirated. Laser scanning microscopy was used to measure cell deformation [DCI (differential interference contrast)] and [Ca²⁺]i throughout the experiment. Ratiometric calcium imaging, as described above, was performed to assess the mechanoresponse of chondrocytes to micropipette aspiration; however, we found that the application of step increases in pressure to the cell surface using a micropipette rarely initiated a Ca²⁺ transient.

Atomic force microscopy—Freshly isolated chondrocytes were plated on glass coverslips and incubated for 2 to 3 days. Before AFM compression, cells were incubated with 10 μM GSK205 or DMSO (vehicle) and stained with intracellular calcium dye Fura-2 AM (Molecular Probes) according to the manufacturer's protocols. Cells were loaded to 400 nN using an AFM (Asylum Research MFP-3D) with tipless cantilevers (k=6.2 N/m; MikroMasch) while simultaneously recording intracellular calcium levels with an Olympus microscope and cycled with 340- and 380-nm light to produce a ratiometric output (Calcium Ratio=IntensityFura-2 @ 380 nm/IntensityFura-2 @ 340 nm) for intracellular calcium levels, which were analyzed with ImageJ.

Pharmacologic TRPV4 modulation—GSK1016790A (GSK101; MilliporeSigma) was resuspended at final concentrations (1 to 10 nM) and matched with a DMSO (vehicle) control. GSK205 (manufactured at the Duke Small Molecule Synthesis Facility) was used as a TRPV4-specific inhibitor and preincubated with samples before analysis to allow diffusion within three-dimensional tissues and used at a final concentration of 10 μM with appropriate DMSO (vehicle) controls.

FLIPR assay—After digestion and isolation, filtered chondrocytes were plated in a 96-well plate at 10,000 cells per well and left for 24 hours before stimulation on a fluorescent imaging plate reader (FLIPR) by which individual wells were stimulated with either osmotic or GSK101-containing medium. Cellular response in each well was measured via fluo-4 intracellular calcium dye using the Fluo-4 No-Wash Calcium Assay Kit (Molecular Probes) according to the manufacturer's directions. GSK205 was preincubated and added alongside stimulated and unstimulated chondrocytes to directly assess the TRPV4-dependent response of osmotic and pharmacologic stimulation.

Finite element modeling: Finite element models of cellular deformation were performed using the FEBio (febio.org) finite element software package (version 2.6.4). Models were run using a neo-Hookean elastic material for the cell and membrane compartments of the cell. All model geometries use axisymmetry boundary conditions to reduce the model size. Osmotic loading was assessed using a Donnan osmotic loading material with parameters taken from the van't Hoff relation for chondrocytes under osmotic loading and loading chondrocytes with a −60 mOsm osmotic medium shift. Models for micropipette aspiration were run assuming a cell modulus of 1 kPa and Poisson's ratio of 0.4 while imposing a pressure of −200 Pa to the cell (61). Models of single-cell direct deformational loading (AFM) were performed by simulating an elastic sphere being compressed to 13% of its original height. FEBio testing suites were used to validate the shell, contact, and neo-Hookean code features, and the Donnan model was validated against the van′t Hoff equation.

Microarray collection and analysis: After a 14-day preculture, engineered cartilage constructs (04 mm×2.25 mm) were stimulated under 10% compressive loading or 1 nM GSK101 for 3 hours per day for 3 days. Unstimulated controls (free-swelling) constructs were cultured under identical conditions. Immediately after stimulation, constructs were washed and then fed with culture medium. Constructs were snap-frozen in liquid N2 at 0, 3, 6, 12, 20, 24, and 72 hours after initial stimulation. Total RNA was extracted from the constructs using a pestle homogenizer and the Norgen Biotek RNA/protein purification plus kit. RNA quantity and quality were assessed with NanoDrop (Thermo Fisher Scientific) and the Agilent Bioanalyzer. Total RNA was processed using the Ambion WT expression labeling kit and the Porcine Gene 1.0 ST Array (Affymetrix). The raw signal of arrays was induced into R environment and quantile-normalized by using “affy” and “oligo” package. The significantly DEGs were identified and analyzed by using one regression model in R with package “genefilter,” “limma,” “RUV,” “splines,” “gplots,” and “plotly” at an adjusted P value cutoff of 0.05. Then, the DEGs were imported into Ingenuity Pathway Analysis (IPA) to perform the pathway enrichment analysis. The DEG heatmap was plotted by using gplots in R.

Mechanogenetic circuit design, development, viral development, and culture: Two lentiviral systems were developed consisting of an NF-κB-inducible promoter upstream of either IL-1Ra (NFKBr-IL1Ra) or luciferase (NFKBr-Luc). Therefore, upon NF-κB signaling, either IL-1Ra (NFKBr-IL1Ra) or luciferase (NFKBr-Luc) is expressed as a measure of mechanogenetic circuit activation. In addition, we developed two lentiviral systems consisting of a synthetic human PTGS2 promoter upstream of either IL-1Ra (PTGS2r-IL1Ra) or luciferase (PTGS2r-Luc). Therefore, when PTGS2 is activated, either IL-1Ra or luciferase is expressed.

NFKBr circuit design: A synthetic NF-κB-inducible promoter was designed to incorporate multiple NF-κB response elements as previously described. A synthetic promoter was developed containing five consensus sequences approximating the NF-κB canonical recognition motif based on genes up-regulated through inflammatory challenge: InfB1, II6, Mcp1, Adamts5, and Cxcl10. A TATA box derived from the minimal CMV promoter was cloned between the synthetic promoter and downstream target genes, either murine II1rn or firefly luciferase from the pGL3 basic plasmid (Promega), and an NF-κB—negative regulatory element was cloned upstream of the promoter to reduce background signal.

PTGS2r circuit design: A synthetic human PTGS2 promoter was obtained from SwitchGear Genomics and cloned into the NFKBr-IL1Ra or NFKBr-Luc lentiviral transfer vectors in place of the NF-κB-inducible promoter. The NF-κB-inducible promoter was excised using Eco RI and Psp XI restriction enzymes, and the PTGS2 promoter was inserted in its place using the Gibson Assembly method to create the PTGS2r-IL1Ra and PTGS2r-Luc circuits.

Lentivirus production and chondrocyte transduction: Human embryonic kidney (HEK) 293T cells were cotransfected with second-generation packaging plasmid psPAX2 (no. 12260; Addgene), the envelope plasmid pMD2.G (no. 12259; Addgene), and the expression transfer vector by calcium phosphate precipitation to make vesicular stomatitis virus glycoprotein pseudotyped lentivirus (66). The lentivirus was harvested at 24 and 48 hours after transfection and stored at −80° C. until use. The expression transfer vectors include the NFKBr-IL1Ra, NFKBr-Luc, PTGS2r-IL1Ra, and PTGS2r-Luc plasmids. The functional titer of the virus was determined with real-time qPCR to determine the number of lentiviral DNA copies integrated into the genome of transduced HeLa cells. For chondrocyte transductions, freshly isolated chondrocytes were plated in monolayer at a density of 62,000 cells/cm² and incubated overnight in standard 10% FBS medium. The following day, virus was thawed on ice and diluted in 10% FBS medium to obtain the desired number of viral particles to achieve a multiplicity of infection of 3. Polybrene was added to a concentration of 4 μg/ml to aid in transduction. The conditioned medium of the chondrocytes was aspirated and replaced with the virus-containing medium, and cells were incubated for an additional 24 hours before aspirating the viral medium and replacing with standard 10% FBS medium. Five days later, cells were trypsinized, counted, and cast in agarose as described above to prepare mechanogenetic constructs at 15 million cells/ml in 2% agarose gel. Constructs were cultured as described above until testing. Viral titers measured by qPCR revealed that ˜95% of chondrocytes were transduced with this method.

Mechanogenetic circuit testing outcome measures: Assessing mechanogenetic tissue construct activation in IL-1Ra-producing constructs was measured with an enzyme-linked immunosorbent assay for mouse IL-1Ra (R&D Systems) according to the manufacturer's protocols. Data are reported as the amount of IL-1Ra produced per construct (in nanograms) normalized by the tissue wet weight mass of the construct (in grams). Luciferase-based mechanogenetic protection was assessed using a bioluminescent imaging reader (Cytation 5, BioTek) at 37° C. and 5% CO₂ and cultured in phenol red-free high-glucose DMEM supplemented with 1% ITS+, 2 mM GlutaMAX, 1 mM sodium pyruvate, 15 mM Hepes, proline (40 μg/ml), 1× penicillin/streptomycin, and 1 μM luciferin. Before stimulation, samples were imaged under free-swelling conditions for ˜1 day to get a baseline bioluminescent level (Fo). For mechanical loading studies, constructs were then mechanically loaded or similarly transferred for a free-swelling control and then returned to the bioluminescent imaging. For GSK101 pharmacologic studies, the bioluminescent medium (above) was supplemented with GSK101 or DMSO (vehicle control) to the appropriate dose (1 to 10 nM) and simultaneously imaged for 3 hours, before washing and replacing with standard bioluminescent medium. Prestimulation baseline was normalized from post-stimulation bioluminescent readings (F) to yield F/Fo as the outcome measure.

Coculture studies: To assess mechanogenetic anti-inflammatory protection, NFKBr-IL1Ra constructs were cultured with a porcine cartilage explant for 72 hours in the presence of porcine IL-1α (0 or 0.1 ng/ml). Porcine cartilage explants (3 mm diameter) were cored from condyle cartilage, and the subchondral bone was removed, leaving a cartilage explant ˜1 to 2 mm thick including the superficial, middle, and deep zones and cultured in iso-osmotic ITS+ medium (380 mOsm, formulation listed above) base medium until experimentation. Mechanical loading was applied daily with hypo-osmotic loading (3 hours/day) (180 mOsm) before returning to the iso-osmotic medium (380 mOsm) containing the explant and IL-1α. Iso-osmotic controls were moved similarly to an iso-osmotic medium (380 mOsm). Control, non-anti-inflammatory mechanogenetic tissue constructs consisted of NFKBr-Luc tissues. Tissue S-GAG content in the engineered tissue construct and medium were assessed with the DMB (methylmethylene blue) assay. Tissue samples were digested with papain overnight (65° C.) to measure tissue S-GAG using the DMB assay. Bulk S-GAG amount was normalized to tissue wet weight. Constructs were fixed in neutral buffered formalin overnight before embedding in paraffin and sectioning to 7 μm. Histological slices were stained with safranin-O to examine S-GAG distribution and abundance.

### Example 2: Mechanogenetic Circuit Development Based on TRPV4 Mechanosensor

RNA-sequencing was used to find gene regulatory targets that are downstream to TRPV4 activation and are not regulated by inflammation. A number of targets are activated by TRPV4 but not modulated by inflammation, demonstrating their selective mechanosensitivity. Circuits were developed based on TRPV4 regulation of FGF1 which required using the endogenous FGF1 promotor for TRPV4-driven activation. These studies identified genes that can be used to differentially regulate smart-cell responses to specific stimuli—in this case in response to inflammation vs mechanical loading.

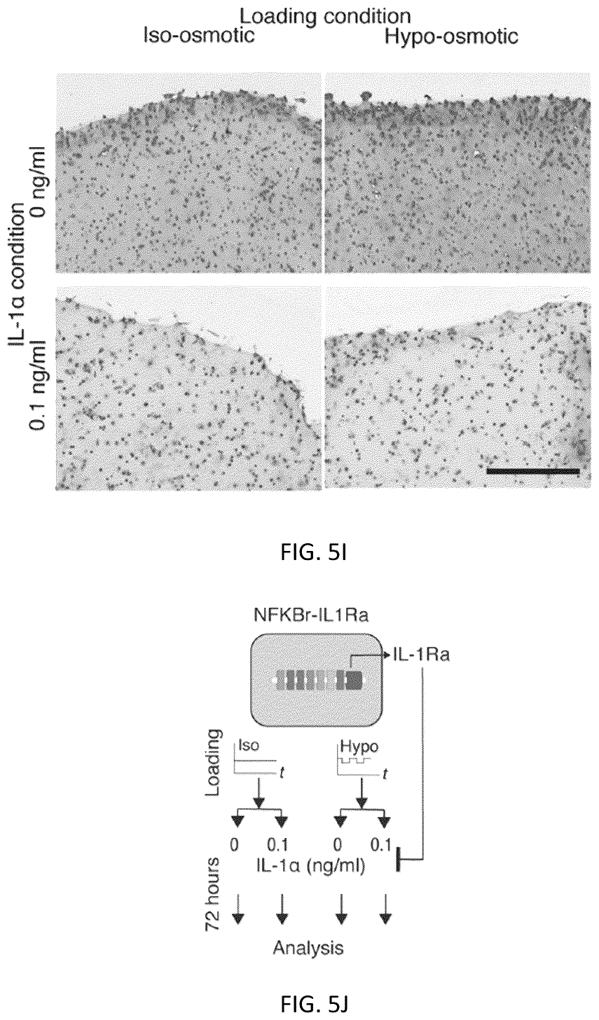

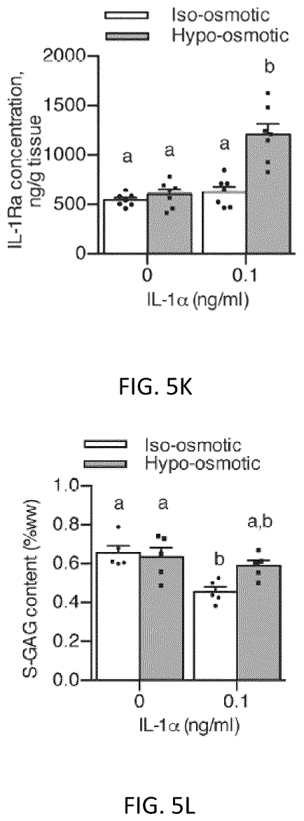

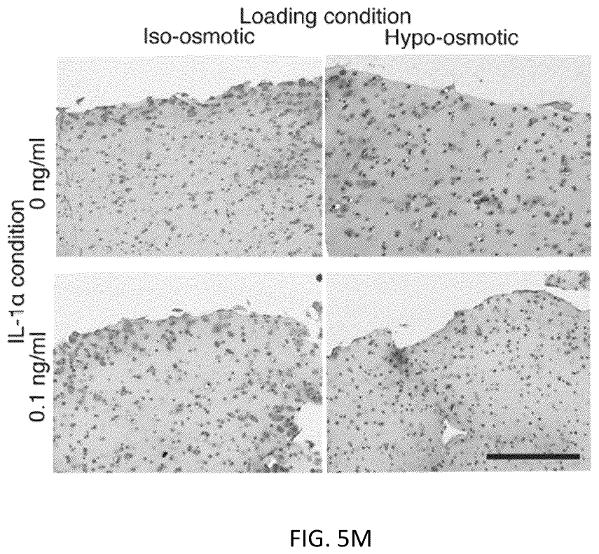

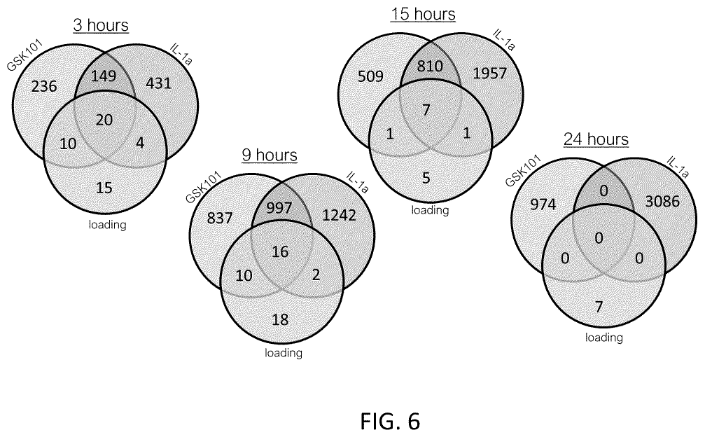

RNA-sequencing was performed to assess mechanically-sensitive gene targets that are distinct from a cellular inflammation response. The data show the engineered cartilage response to mechanical loading, GSK101 agonist activation of TRPV4, and IL-1α inflammation (see FIG. 6). Targets of interest were selected as targets that were shared between stimulation to mechanical loading and GSK101 agonist activation of TRPV4 but not stimulated by IL-1α inflammation.

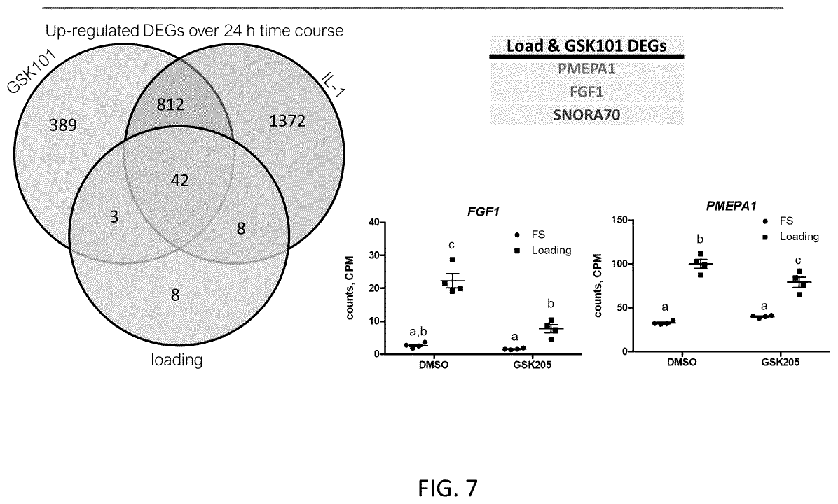

Gene targets fibroblast growth factor 1 (FGF1), prostate transmembrane androgen induced 1 (PMEPA1), and small nucleolar RNA 70 (snora70) were significantly upregulated by mechanical loading and GSK101 TRPV4 activation, but not IL-1α inflammation over a 24 hour time course as assessed by RNA-sequencing of tissue engineered cartilage. The mechanical loading response of FGF1 and PMEPA1 were inhibited by the TRPV4 inhibitor GSK205, demonstrating the TRPV4 regulation of these targets (see FIG. 7).

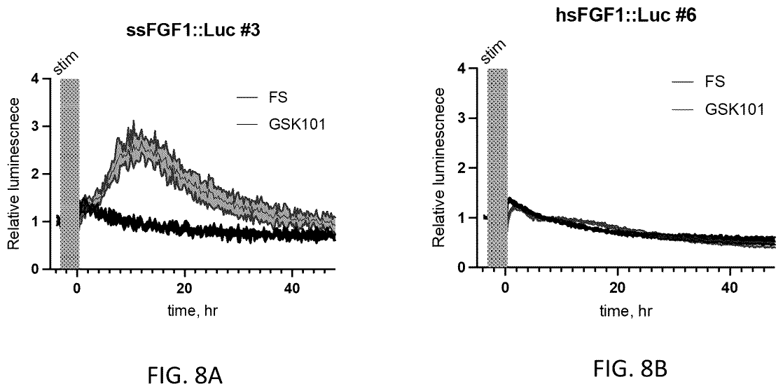

Mechanogenetic circuits were engineered using the endogenous porcine FGF1 promotor (˜2 kb, FIG. 8 left graph, ssFGF1::Luc #3) or a synthetic FGF1 promotor from the human genome (˜1 kb, FIG. 8 right graph, hsFGF1::Luc #6). TRPV4 was activated by GSK101 stimulation for 3 hours (GSK101, blue trace), or using a DMSO vehicle control (FS, black trace). Circuits made with the endogenous FGF1 promotors were activated by GSK101 stimulation, while mechanogenetic circuits using synthetic human promotors were not responsive to TRPV4 activation (see FIG. 8).

### Example 3: Mechanogenetic Circuit Development Based on Piezo1 Mechanosensor

RNA-sequencing was used to find gene regulatory targets that are downstream to Piezo1 activation. A number of targets are activated by Piezo1 using the Piezo1 agonist, Yoda1, and the response to high levels of mechanical loading (80% strain), demonstrating their selective mechanosensitivity to Piezo1 activation. These studies identified genes that can be used to differentially regulate smart-cell responses to specific stimuli—in this case in response to specific to different ion channels (TRPV4 vs Piezo) activated by different levels of loading.

Tissue engineered cartilage tissues were either mechanically loaded to 80% strain or stimulated with the Piezo1 agonist Yoda1. Control tissues (FS group) were cultured in vehicle control media and left under free-swelling (non loaded) conditions. RNA-seq was used to assess the transcriptomic regulation to high mechanical loading or Yoda1-stimulation.

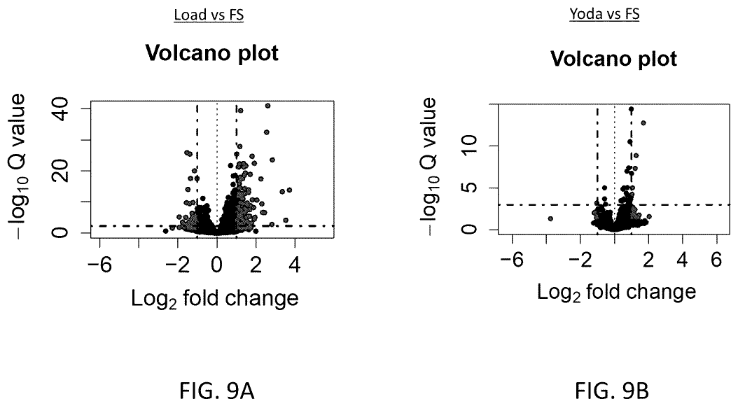

Volcano plot projections of gene targets that are differentially regulated by mechanical loading to 80% strain (FIG. 9 left graph) or Yoda1 stimulation (FIG. 9 right graph) display a range of targets that are regulated by Piezo1 stimulation (targets highlighted in red are |LFC|>1 and FDR (padj)<0.05).

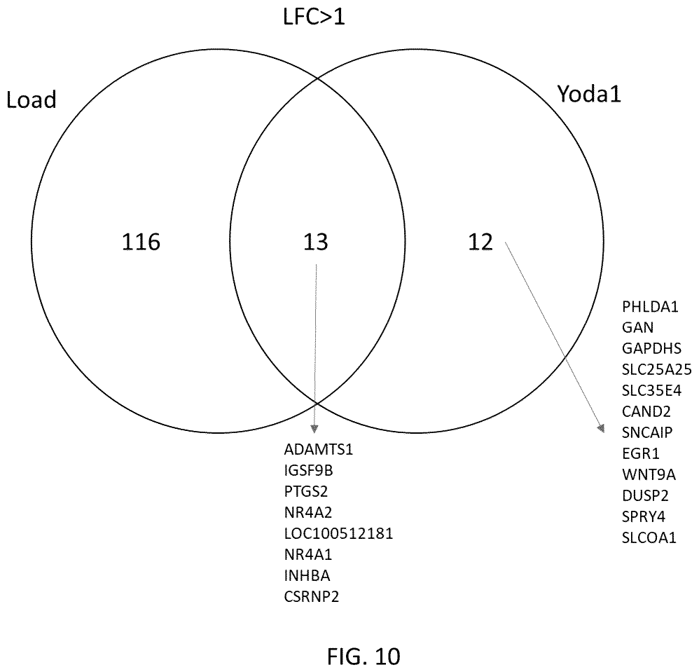

Gene regulation targets that were similarly regulated by mechanical loading and Yoda1 stimulation demonstrated targets that are downstream to Piezo1 stimulation and include ADAMTS1, IGSF9B, PTGS2, NR4A2, LOC100512181, NR4A1, INHBA, and CSRNP2. These represent targets for mechanogenetic circuits that are regulated by Piezo1 and which can be used to distinguish the response driven by TRPV4 (see FIG. 10).

1. A recombinant nucleic acid sequence comprising at least one transcriptional regulatory nucleic acid sequence of a mechanically-responsive gene operably linked to a nucleic acid sequence encoding a therapeutic biologic.

2. The nucleic acid sequence of claim 1, wherein the at least one transcriptional regulatory nucleic acid sequence nucleic acid sequence is a promoter nucleic acid molecule of a mechanically-responsive gene.

3. The nucleic acid sequence of claim 1, wherein the nucleic acid sequence further comprises a TATA box between the at least one transcriptional regulatory nucleic acid sequence of a mechanically-responsive gene and the nucleic acid sequence encoding a therapeutic biologic.

4. A nucleic acid construct or vector comprising the nucleic acid sequence of claim 1.

5. The vector of claim 4, wherein the vector is a viral vector.

6. The viral vector of claim 5, wherein the viral vector is an adeno-associated viral vector or a lentiviral vector.

7. (canceled)

8. The nucleic acid claim 2, wherein
the promoter of a mechanically-responsive gene is selected from a NFKB promoter, a PTGS2 promoter, a PMEPA1 promoter, a FGF1 promoter, a SNORA70 promoter, a ADAMTS1 promoter, a IGSF9B promoter, a NR4A2 promoter, a NR4A1 promoter, a INHBA promoter, a CSRNP2 promoter, a PHLDA1 promoter, a GAN promoter, a SLC25A25 promoter, a SLC35E4 promoter, a CAND2 promoter, a SNCAIP promoter, a WNT9A promoter, a EGR1 promoter, a DUSP2 promoter, a SPRY4 promoter and combinations thereof.

9. The nucleic acid claim 1, wherein the therapeutic biologic nucleic acid sequence encodes an anti-catabolic polypeptide, an anti-inflammatory polypeptide, a pro-anabolic polypeptide, a pro-regenerative polypeptide, an anti microbial polypeptide, an anti-pain polypeptide, a morphogen, a growth factor, an anti cancer nucleic acid or an anti-cancer polypeptides.

10. The nucleic acid of claim 9, wherein the promoter is a PTGS2 promoter and the therapeutic biologic is an IL-1 receptor antagonist.

11. A genetically modified cell comprising a heterologous nucleic acid sequence incorporated into its genome, wherein the heterologous nucleic acid sequence comprises the nucleic acid sequence of claim 1.

12.-16. (canceled)

17. The genetically modified cell of claim 11, wherein the cell expresses on its surface a mechanically sensitive ion channel.

18. The genetically modified cell of claim 17, wherein the mechanically sensitive ion channel is from the transient receptor potential (TRP) family.

19. The genetically modified cell of claim 18, wherein the mechanically sensitive ion channel is selected from TRPA1, TRP vanilloid 1 (TRPV1), and TRPV4.

20. The genetically modified cell of claim 19, wherein the mechanically sensitive ion channel is TRPV4.

21.-22. (canceled)

23. The genetically modified cell of claim 17, wherein the mechanically sensitive ion channel is PIEZ01 and/or PIEZ02.

24. The genetically modified cell of claim 23, wherein the promoter of a mechanically-responsive gene is selected from a ADAMTS1 promoter, a NR4A2 promoter, a NR4A1 promoter, a WNT9A promoter, a SPRY4 promoter, and combinations thereof.

25. The genetically modified cell of claim 11, wherein the therapeutic biologic nucleic acid molecule encodes for one or more of IL-1 Ra, sTNFR1/2, IL-10 IL-4, a growth factor from the TGF superfamily, IGF, CTGF, FGF, PDGF, a kappa opioid ligand pro-peptide (e.g., prodynorphin), a mu/delta opioid ligand pro-peptide (e.g., proenkephalin), a delta/mu opioid ligand pro-peptide (proopiomelanocortin), an endocannabinoid ligand synthesis drivers TNF, IL-7, IL-15, IL-12, IL-2, IFN, NOS, PTGIS, Decorin, TGF-receptor, MMP, ALDH2, NR3C1 and any combination thereof.

26.-27. (canceled)

28. The genetically modified cell of claim 11, wherein the period, frequency, or phase of the therapeutic biologic expression is modulated through the at least one transcriptional regulatory nucleic acid molecule of a mechanically responsive gene or through use of combinations of the transcriptional regulatory nucleic acid molecules of a mechanically responsive gene.

29.-31. (canceled)

32. A method of treating a condition, disease or disorder in a subject in need thereof, the method comprising administering an effective amount of a composition comprising the genetically modified cell of claim 11.

33. The method of claim 32, wherein the condition, disease or disorder is a chronic inflammatory disease, an acute inflammatory disease, a degenerative disease of any tissue or organ, chronic or acute pain, cancer, cardiovascular disease, osteoarthritis, cardiac hypertrophy, atherosclerosis, asthma, irritable bowel syndrome, muscle atrophy, angina, atrial fibrillation, hypertension, intimal hyperplasia, valve disease, scleroderma, achalasia, volvulus, diabetic nephropathy, glomerulosclerosis, cerebral edema, hydrocephalus, migraine, stroke, glaucoma, ankylosing spondylitis, carpal tunnel syndrome, chronic back pain, dupytre's contracture, osteoporosis, rheumatoid arthritis, collagenopathies, Musculo dystrophies, osteochondroplasias, polycystic kidney disease, ARDS, emphysema, pulmonary fibrosis, ventilator injury, pre-eclampsia, sexual dysfunction, or urinary incontinence.
